(19) United States
(12) Patent Application Publication
(10) Pub. No.: US 2011/0311613 A1
(43) Pub. Date: Dec. 22, 2011

(54) TOPICAL FORMULATIONS OF FLAP INHIBITORS FOR THE TREATMENT OF DERMATOLOGICAL CONDITIONS

(75) Inventors: John Howard Hutchinson, San Diego, CA (US); Jillian F. Evans, Rancho Santa Fe, CA (US); Kevin Murray Schaab, Spring Valley, CA (US)

(73) Assignee: AMIRA PHARMACEUTICAL, INC., San Diego, CA (US)

(21) Appl. No.: 13/141,665

(22) PCT Filed: Dec. 21, 2009
(86) PCT No.: PCT/US2009/069065

§ 371 Date: Sep. 12, 2011

Related U.S. Application Data
(60) Provisional application No. 61/140,522, filed on Dec. 23, 2008.

(51) Int. Cl. A61K 9/127; A61K 31/444; A61K 31/4709; A61K 31/405; A61K 31/47; A61K 31/56; A61K 38/13; A61P 17/00; A61P 35/00; A61P 37/00; A61P 37/08; A61P 17/06; A61P 17/04; A61P 17/02; A61P 17/10; A61K 31/506; B82Y 5/00

(52) U.S. Cl. 424/450; 514/269; 514/255.05; 514/333; 514/314; 514/418; 514/311; 514/414; 514/171; 514/18.6; 977/773; 977/915

(57) ABSTRACT

Described herein, are topical formulations for treating a dermatological disease, disorder, or condition. Topical formulations disclosed herein include a therapeutically-effective amount of a FLAP inhibitor formulated for dermal administration

### RELATED APPLICATIONS

This application claims benefit of U.S. Provisional Application No. 61/140,522, entitled “TOPICAL FORMULATIONS OF FLAP INHIBITORS FOR THE TREATMENT OF DERMATOLOGICAL CONDITIONS” filed on Dec. 23, 2008, which is herein incorporated by reference.

### FIELD OF THE INVENTION

Described herein are pharmaceutical compositions for topical administration to the skin of a mammal that include at least one 5-lipoxygenase activating protein (FLAP) inhibitor compound and methods of use thereof in the treatment or prevention of dermal diseases, disorders or conditions.

### BACKGROUND OF THE INVENTION

Topical administration of a 5-lipoxygenase activating protein (FLAP) inhibitor compound is used to treat dermal diseases, disorders or conditions. Dermal diseases, disorders or conditions include, but are not limited to, acne, psoriasis, dermatitis (e.g., contact or atopic), eczema, urticaria, rosacea, burns, and scarring. In certain instances, dermal diseases, disorders or conditions result from an over-production of leukotrienes.

### SUMMARY OF THE INVENTION

Described herein, in certain embodiments, are topical formulations for treating a dermatological disease, disorder or condition (i.e., an abnormal state of the epidermis, dermis, and/or subcutaneous tissues). Described herein, in certain embodiments, are topical formulations for treating an immune disorder (e.g. an autoimmune disorder (e.g., eczema, psoriasis)); a proliferative disorder (e.g., melanoma); contact with an allergen, and/or an irritant; an overproduction of sebum lipids (e.g., acne); (e.g., scarring, including acne scarring); a burn (e.g., first degree, second degree, third degree, or fourth degree); or combinations thereof. Described herein, in certain embodiments, are topical formulations for treating a chronic blistering disorder, acne, psoriasis, dermatitis (e.g., contact or atopic), eczema, urticaria, rosacea, scarring (i.e. the formation of a scar (e.g., a keloid scar or a hypertrophic scar)), a first degree burn, a second degree burn, a third degree burn, a fourth degree burn and/or melanoma. In some embodiments, a topical formulation disclosed herein comprises a therapeutically-effective amount of a FLAP inhibitor compound. In some embodiments, a topical formulation disclosed herein is administered before or after contact with an allergen and/or irritant. In some embodiments, a topical formulation disclosed herein is administered before or after a physical trauma (e.g., surgery).

In one aspect, described herein is a topical formulation comprising a FLAP inhibitor compound in an amount effective for the treatment of a dermatological disease, disorder or condition, and suitable excipients to provide an ointment, cream, lotion, paste, gel, stick, a liposome, a nanoparticle, a patch or wound dressing.

In some embodiments, the FLAP inhibitor compound inhibits leukotriene synthesis, antagonizes a leukotriene receptor, inhibits Interleukin-4 (IL-4) synthesis, or inhibits or reduces mucin synthesis.

In some embodiments, the dermatological disease, disorder or condition is an immune disorder; a proliferative disorder; contact with an allergen and/or an irritant, itching, atopic dermatitis, allergic dermatitis, bullous disorders, collagenoses, psoriasis, psoriatic lesions, contact dermatitis, eczema, urticaria, rosacea, hypertrophic scarring, keloid scar formation, scleroderma, Folliculitis keloidalis nuchae, Kawasaki Disease, Sjogren-Larsso Syndrome, Grover's disease, an overproduction of sebum lipids, acne, a first degree burn, a second degree burn, a third degree burn, a fourth degree burn, solar keratosis, squamous cell carcinoma or melanoma or combinations thereof.

In some embodiments, the topical formulation further comprises a therapeutically-effective amount of an second compound selected from antibiotics; anti-fungal agents; steroid anti-inflammatory agents; non-steroidal anti-inflammatory agents; antihistamines; antivirals; alpha agonists; beta blockers; carbonic anhydrase inhibitors; miotics; prostaglandins; anti-angiogenesis agents; loteprednol etabonate, mast cell stabilizers, cyclosporine, and DP2 antagonists. In certain embodiments, the topical formulation further comprises a therapeutically-effective amount of a DP₂ receptor antagonist.

In one aspect, described herein is a method of treating or preventing a dermatological disease, disorder or condition, comprising administering to an individual in need thereof a therapeutically-effective amount of a topical formulation comprising a FLAP inhibitor compound.

In some embodiments of the method, the FLAP inhibitor compound inhibits leukotriene synthesis, antagonizes a leukotriene receptor, inhibits Interleukin-4 (IL-4) synthesis, or inhibits mucin synthesis. In some embodiments, the topical formulation is in the form of an ointment, cream, lotion, paste, gel, stick, a liposome, a nanoparticle, a patch or wound dressing.

In some embodiments of the method, the dermatological disease, disorder or condition is an immune disorder; a proliferative disorder; contact with an allergen and/or an irritant, itching, atopic dermatitis, allergic dermatitis, bullous disorders, collagenoses, psoriasis, psoriatic lesions, contact dermatitis, eczema, urticaria, rosacea, hypertrophic scarring, keloid scar formation, scleroderma, Folliculitis keloidalis nuchae, Kawasaki Disease, Sjogren-Larsso Syndrome, Grover's disease, an overproduction of sebum lipids, acne, a first degree burn, a second degree burn, a third degree burn, a fourth degree burn, solar keratosis, squamous cell carcinoma or melanoma or combinations thereof.

In certain embodiments of the method, the dermatological disease, disorder or condition is scarring. In certain instances, the scarring results in the formation of a keloid scar.

In certain embodiments of the method, the dermatological disease, disorder or condition results from surgery. In certain instances, the topical formulation is administered before surgery. In certain instances, the topical formulation is administered after surgery.

In certain embodiments of the method, the topical formulation is administered before contact with an irritant and/or allergen. In certain instances, the topical formulation is administered after contact with an irritant and/or allergen.

In certain embodiments, provided is a method for the treatment or prevention of itching in a mammal comprising administering to the mammal a topical formulation described herein comprising a therapeutically-effective amount of a FLAP inhibitor compound. In some embodiments, the itching is a symptom of any of the diseases or conditions described herein. In some embodiments, the itching is caused by contact with an irritant, allergen, or combination thereof. In some embodiments, the itching is a symptom of dermatitis, eczema, urticaria, or psoriasis. In some embodiments, the itching is a symptom of atopic dermatitis or allergic dermatitis.

In one aspect, provided herein is a method for the treatment or prevention of a rash in a mammal comprising administering to the mammal a topical formulation described herein comprising a therapeutically-effective amount of a FLAP inhibitor compound. In some embodiments, the rash is a symptom of any of the diseases or conditions described herein. In some embodiments, the rash is caused by contact with an irritant, allergen, or combination thereof. In some embodiments, the rash is a symptom of dermatitis, eczema, urticaria, or psoriasis. In some embodiments, the rash is a symptom of atopic dermatitis or allergic dermatitis.

In one aspect, provided herein is a method for the treatment or prevention of skin inflammation in a mammal comprising administering to the mammal a topical formulation described herein comprising a therapeutically-effective amount of a FLAP inhibitor compound. In some embodiments, the skin inflammation is a symptom of any of the diseases or conditions described herein. In some embodiments, the skin inflammation is caused by contact with an irritant, allergen, or combination thereof. In some embodiments, the skin inflammation is a symptom of dermatitis, eczema, urticaria, or psoriasis. In some embodiments, the skin inflammation is a symptom of atopic dermatitis or allergic dermatitis.

In one aspect, provided herein is a method for the treatment or prevention of blisters, redness, swelling, scabbing, scaling, or combinations thereof in a mammal comprising administering to the mammal a topical formulation described herein comprising a therapeutically-effective amount of a FLAP inhibitor compound. In some embodiments, the blisters, redness, swelling, scabbing, scaling, or combinations thereof is a symptom of any of the diseases or conditions described herein. In some embodiments, the blisters, redness, swelling, scabbing, scaling, or combinations thereof is caused by contact with an irritant, allergen, or combination thereof. In some embodiments, the blisters, redness, swelling, scabbing, scaling, or combinations thereof is a symptom of dermatitis, eczema, urticaria, or psoriasis. In some embodiments, the blisters, redness, swelling, scabbing, scaling, or combinations thereof is a symptom of atopic dermatitis or allergic dermatitis.

In some embodiments of the method, the administration further comprises a therapeutically-effective amount of an second compound selected from antibiotics; anti-fungal agents; steroid anti-inflammatory agents; non-steroidal anti-inflammatory agents; antihistamines; antivirals; alpha agonists; beta blockers; carbonic anhydrase inhibitors; miotics; prostaglandins; anti-angiogenesis agents; loteprednol etabonate, mast cell stabilizers, cyclosporine, and DP₂ antagonists. In certain embodiments, the administration further comprises a therapeutically-effective amount of a DP₂ antagonist.

In one aspect is the use of a FLAP inhibitor compound in the manufacture of a topical formulation for application to the skin. In one aspect is the use of a combination of a FLAP inhibitor compound and a second therapeutic agent in the manufacture of a topical formulation for application to the skin.

In one aspect is the use of a FLAP inhibitor compound in the manufacture of a topical formulation for the treatment of a dermal disease, disorder or condition. In one aspect is the use of a FLAP inhibitor compound and a second therapeutic agent in the manufacture of a topical formulation for the treatment of a dermal disease, disorder or condition.

### BRIEF DESCRIPTION OF THE FIGURES

FIG. 1 presents illustrative examples of FLAP inhibitor compounds described herein.

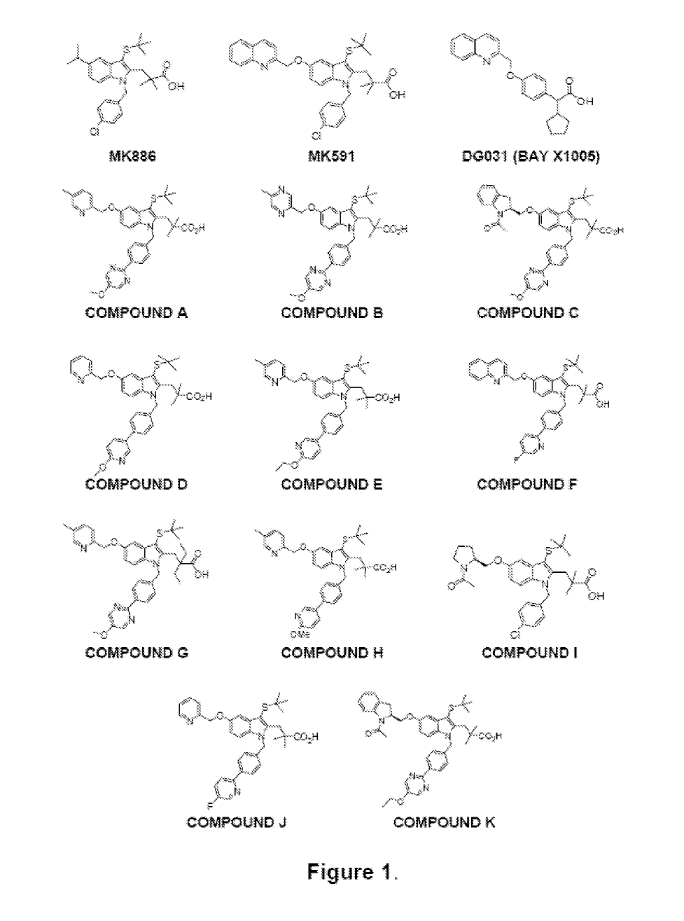

FIG. 2 illustrates the effect of the orally administered FLAP inhibitor Compound A on ear inflammation, LTB₄ and CysLT levels resulting from arachidonic acid-induced ear inflammation.

FIG. 3 illustrates the effect of the dermally administered FLAP inhibitor Compound A on ear inflammation, LTB₄ and CysLT levels resulting from arachidonic acid-induced ear inflammation.

FIG. 4 illustrates the effect of FLAP inhibition and DP₂ receptor antagonism on the number of total cells, neutrophils and lymphocytes present in bronchioalveolar lavage fluid (BALF).

FIG. 5 illustrates the effect of a combination of a FLAP inhibitor and a DP₂ receptor antagonist on the presence of mucin in BALF.

### DETAILED DESCRIPTION OF THE INVENTION

Leukotrienes are a class of pro-inflammatory lipid mediators derived from arachidonic acid that have been shown to play important roles in a number of biological processes. Arachidonic acid is converted to leukotriene A₄ (LTA₄) in a two-step process mediated by the enzyme 5-lipoxygenase (5-LO). LTA₄ is converted either to LTB₄ via LTA₄ hydrolase or to LTC₄ through conjugation with glutathione mediated by LTC₄ synthase. Amide bond cleavage converts LTC₄ to LTD₄ and then subsequently to LTE₄. The initial oxidation step is a process that requires the intimate involvement of both 5-LO and the membrane bound 5-lipoxygenase-activating protein (FLAP). Inhibition of FLAP results in the inhibition of all leukotriene production. LTB₄ is the ligand for the G protein-coupled receptors (GPCRs) BLT₁ and BLT₂ and both receptors are involved in chemotaxis and cell stimulation in the inflammatory response.

Leukotrienes are lipid mediators of inflammation that are involved in the pathogenesis of dermatological diseases, disorders or conditions. Leukotrienes are produced mainly by mast cells, eosinophils, monocytes/macrophages, and neutrophils in response to allergic or inflammatory stimuli. In one aspect, biological tissues in areas that are affected by a dermal condition have high levels of leukotrienes. The role of FLAP in the leukotriene synthesis pathway is significant because FLAP in concert with 5-lipoxygenase performs the first step in the pathway for the synthesis of leukotrienes Inhibiting FLAP provides a target for the treatment of leukotriene-dependent or leukotriene mediated dermatological diseases, disorders or conditions, including, by way of example, immune diseases, disorders or conditions, (e.g. autoimmune diseases, disorders or conditions (e.g., eczema, psoriasis)); proliferative conditions (e.g., melanoma); contact with an allergen and/or an irritant; a mast cell diseases or conditions; scarring (e.g., scarring after a trauma (e.g., surgery)); burns; inflammatory diseases or conditions affecting the skin, or combinations thereof.

Disclosed herein is the use of FLAP inhibitors in the manufacture of medicaments suitable for topical administration to a mammal for the treatment or prevention of leukotriene-dependent or leukotriene mediated dermatological diseases, disorders or conditions.

Described herein are pharmaceutical compositions suitable for topical administration, methods for treating, methods for preventing, methods for formulating topical formulations, methods for producing, methods for manufacturing, treatment strategies, using FLAP inhibitors.

Described herein, in certain embodiments, are topical formulations that include a FLAP inhibitor compound for treating a dermatological disease, disorder or condition. In one aspect, topical administration of a FLAP inhibitor compound to a mammal minimizes systemic absorption of the FLAP inhibitor compound. In one aspect, topical administration of a FLAP inhibitor compound provides for local treatment of a dermal disease, disorder or condition. In one aspect, local treatment of a dermal disease, disorder or condition with a FLAP inhibitor compound reduces possible side effects associated with systemic administration of a FLAP inhibitor compound.

In some embodiments, a topical formulation described herein comprises a FLAP inhibitor compound in combination with an antibiotic; anti-fungal agent; steroid anti-inflammatory agent; non-steroidal anti-inflammatory agent; antihistamine; antiviral; alpha agonist; beta blocker; carbonic anhydrase inhibitor; miotic; prostaglandin; anti-angiogenesis agent; loteprednol etabonate, mast cell stabilizer, cyclosporine, and/or DP2 antagonist to treat or prevent a dermatological disease, disorder or condition.

In one aspect, the dermatological disease, disorder or condition is a result of the over-production of leukotrienes and/or cytokines. In one aspect, the dermatological disease, disorder or condition includes, but is not limited to, dermatological immune diseases, disorders or conditions, dermatological proliferative conditions, a dermatological disease, disorder or condition resulting from contact with an allergen and/or an irritant, a dermatological mast cell disease, disorder or condition, a burn, an inflammatory disease, disorder or condition affecting the skin, or combinations thereof. Allergens and/or irritants include, but are not limited to, uruishol, alcohol, xylene, turpentine, esters, acetone, ketones. Dermatological immune diseases, disorders or conditions include, but are not limited to, eczema, psoriasis. Dermatological proliferative disorders include, but are not limited to, melanoma. Dermatological disorders related to overproduction of sebum lipids include, but are not limited to, acne. Dermatological mast cell disorders include but are not limited to, fibroblast disorders including scarring, such as the formation of keloid scars, hypertrophic scars, and/or acne scars. Dermatological burn disorders include, but are not limited to, a first degree burn, a second degree burn, a third degree burn, or a fourth degree burn.

Described herein, in certain embodiments, are topical formulations that include a FLAP inhibitor compound for treating a chronic blistering disorder, acne, psoriasis, dermatitis (e.g., contact or atopic), eczema, urticaria, rosacea, scarring (i.e. the formation of a scar (e.g., a keloid scar or a hypertrophic scar)), a first degree burn, a second degree burn, a third degree burn, a fourth degree burn and/or melanoma. In some embodiments, a topical formulation disclosed herein comprises a therapeutically-effective amount of a FLAP inhibitor. In some embodiments, a topical formulation disclosed herein is administered before or after contact with an allergen and/or irritant. In some embodiments, a topical formulation disclosed herein is administered before or after a physical trauma (e.g., surgery). In one aspect, a topical formulation disclosed herein that includes a FLAP inhibitor compound is topically administered to treat and prevent scar formation following surgery. It is understood that the topical formulation is applied to the site of injury.

In certain instances, leukotrienes and/or cytokines are involved in scarring and/or the migration of fibroblasts. In one aspect, inhibiting the activity of FLAP inhibits the activity of and/or migration of fibroblasts, and/or treats scarring. In certain instances, inhibiting the activity of FLAP reduces or inhibits the deposition of mucin in the interstitial spaces of the dermis.

In certain instances, leukotrienes mediate the production of lipids from sebaceous glands. In certain instances, inhibiting the synthesis of leukotrienes (e.g. by inhibiting FLAP) reduces the number of inflammatory lesions in moderate acne and inhibits the synthesis of sebaceous lipids.

In one aspect, leukotrienes are involved in the pathogenesis of dermatological diseases, disorders or conditions described herein. In some instances, inhibition of FLAP will result in a decrease in the production of leukotrienes. In some instances, a reduction of leukotrienes results in a decrease of inflammation, and/or fibrosis.

### FLAP Inhibitors

In one aspect, the FLAP inhibitor compound is selected from FLAP inhibitor compounds disclosed herein or in the art.

In one aspect, the FLAP inhibitor compound is a compound of Formula (I), pharmaceutically acceptable salt, pharmaceutically acceptable solvate, or N-oxide thereof:

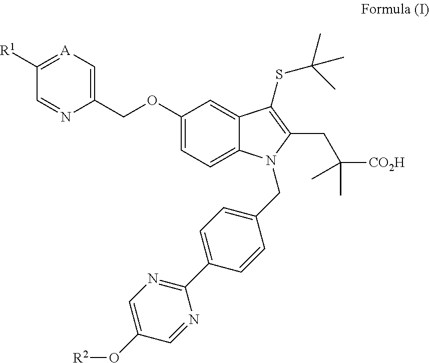

wherein,

### A is CH or N;

R¹ is —F, —Cl, —Br, —CN, C₁-C₄alkyl, C₁-C₄fluoroalkyl, —O—C₁-C₄alkyl, or —O—C₁-C₄fluoroalkyl;
R² is C₁-C₄alkyl or C₁-C₄fluoroalkyl.

In one aspect, A is CH. In another aspect, A is N.

In one aspect, R¹ is —F, —Cl, —Br, —CN, —CH₃, —CH₂CH₃, cyclopropyl, —CF₃, —OCH₃, —OCH₂CH₃, or —OCF₃. In another aspect, R¹ is —F, —Cl, —Br, —CN, —CH₃, —CF₃, —OCH₃, or —OCF₃. In another aspect, R¹ is —CH₃.

In one aspect, R² is C₁-C₄alkyl. In another aspect, R² is —CH₃, or —CH₂CH₃. In another aspect, R² is —CH₃.

In one aspect, R¹ is —CH₃ and R² is —CH₃. In one aspect, A is CH, R¹ is —CH₃, and R² is —CH₃.

In one aspect, the FLAP inhibitor compound is 3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid (Compound A); or 3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid (Compound B). In one aspect, the FLAP inhibitor compound is Compound A.

In another aspect, the FLAP inhibitor compound is a compound of Formula (II), pharmaceutically acceptable salt, pharmaceutically acceptable solvate, or N-oxide thereof:

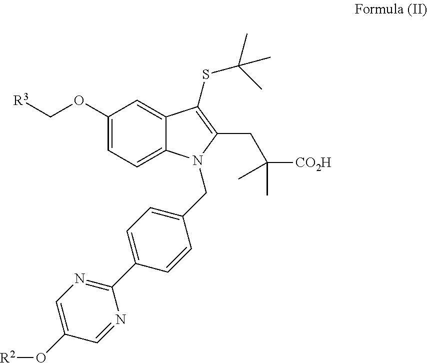

wherein,
R² is C₁-C₄alkyl or C₁-C₄fluoroalkyl;
R³ is a substituted or unsubstituted monocyclic or bicyclic heterocycloalkyl.

In one aspect, R² is C₁-C₄alkyl. In another aspect, R² is —CH₃, or —CH₂CH₃. In another aspect, R² is —CH₃.

In one aspect, R³ is a substituted or unsubstituted monocyclic or bicyclic heterocycloalkyl containing at least one N atom in the ring. In one aspect, R³ is a substituted or unsubstituted monocyclic or bicyclic C₃-C₁₀heterocycloalkyl containing at least one N atom in the ring.

In one aspect, R³ is a substituted or unsubstituted monocyclic or bicyclic heterocycloalkyl selected from quinolizinyl, dioxinyl, piperidinyl, morpholinyl, thiazinyl, tetrahydropyridinyl, piperazinyl, oxazinanonyl, dihydropyrrolyl, dihydroimidazolyl, tetrahydrofuranyl, dihydrooxazolyl, oxiranyl, pyrrolidinyl, pyrazolidinyl, imidazolidinonyl, pyrrolidinonyl, dihydrofuranonyl, dioxolanonyl, thiazolidinyl, piperidinonyl, tetrahydronaphyridinyl, tetrahydroquinolinyl, tetrahydrothienyl, indolinyl, and thiazepanyl.

In one aspect, R³ is a substituted or unsubstituted monocyclic or bicyclic heterocycloalkyl selected from piperidinyl, morpholinyl, thiazinyl, tetrahydropyridinyl, piperazinyl, dihydropyrrolyl, dihydroimidazolyl, pyrrolidinyl, pyrazolidinyl, imidazolidinonyl, pyrrolidinonyl, thiazolidinyl, piperidinonyl, tetrahydronaphyridinyl, tetrahydroquinolinyl, and indolinyl.

In one aspect, R³ is a substituted or unsubstituted monocyclic or bicyclic heterocycloalkyl selected from piperidinyl, morpholinyl, piperazinyl, pyrrolidinyl, pyrrolidinonyl, piperidinonyl, tetrahydroquinolinyl, and indolinyl.

In one aspect, R³ is a substituted or unsubstituted monocyclic or bicyclic heterocycloalkyl selected from piperidinyl, morpholinyl, piperazinyl, pyrrolidinyl, tetrahydroquinolinyl, and indolinyl. In one aspect, R³ is a substituted or unsubstituted pyrrolidinyl, and indolinyl. In one aspect, R³ is a substituted or unsubstituted indolinyl.

In further or alternative embodiments, R³ is selected from the group consisting of:

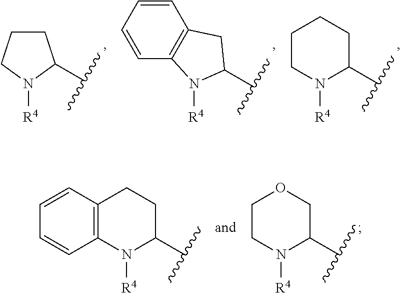

R⁴ is H, —C(═O)R⁵ or —SO₂—C₁-C₄alkyl; R⁵ is C₁-C₄alkyl, C₁-C₄fluoroalkyl, substituted or unsubstituted phenyl, substituted or unsubstituted heteroaryl, or —O—C₁-C₄alkyl.

In further or alternative embodiments, R³ is selected from the group consisting of:

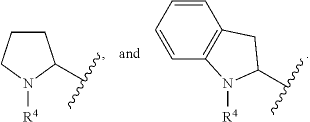

In one aspect, R³ is

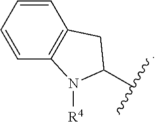

In one aspect, R⁴ is —C(═O)R⁵.

In further or alternative embodiments, R³ is selected from the group consisting of:

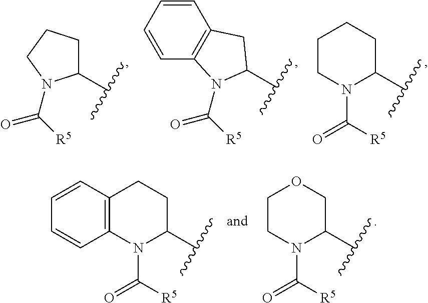

In further or alternative embodiments, R³ is selected from the group consisting of:

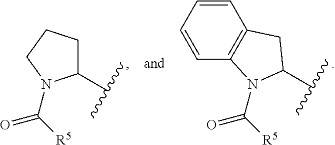

In one aspect, R³ is

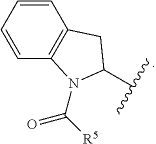

In one aspect, R⁵ is C₁-C₄alkyl, C₁-C₄fluoroalkyl, substituted or unsubstituted phenyl, or —O—C₁-C₄alkyl. In another aspect, R⁵ is —CH₃, CH₂CH₃, —CH₂CH₂CH₃, —CH₂CH₂CH₂CH₃, —CH(CH₃)₂, —C(CH₃)₃, —CF₃, —OCH₃, —OCH₂CH₃, or —OC(CH₃)₃. In another aspect, R⁵ is —CH₃, —CH₂CH₃, —CH₂CH₂CH₃, —CF₃, —OCH₃, —OCH₂CH₃, or —OC(CH₃)₃. In yet another aspect, R⁵ is —CH₃.

In one aspect, the compound of Formula (II) has the following structure:

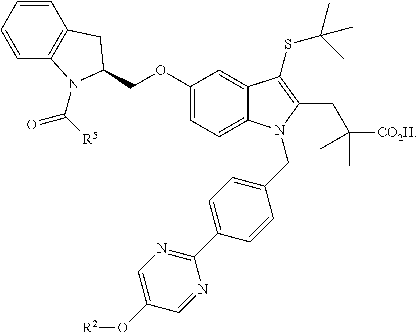

In one aspect, the FLAP inhibitor compound is 3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid (Compound C) or 3-{5-((S)-1-Acetyl-2,3-dihydro-1H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid (Compound K).

In one aspect, the FLAP inhibitor is selected from compounds described in U.S. patent application Ser. No. 11/538,762 (issued as U.S. Pat. No. 7,405,302); U.S. patent application no. 12/131,828; U.S. patent application Ser. No. 11/553,946 (published as 2007/0105866); U.S. patent application Ser. No. 11/925,841; U.S. patent application Ser. No. 12/089,706; U.S. patent application Ser. No. 12/089,707; U.S. patent application Ser. No. 12/092,570; U.S. patent application Ser. No. 11/744,555 (published as 2007/0219206); U.S. patent application Ser. No. 11/746,010 (published as 2007/0225285); U.S. patent application Ser. No. 11/745,387 (published as 2007/0244128); U.S. patent application Ser. No. 12/257,876; U.S. patent application No. 61/055,887; U.S. patent application No. 61/055,899; International Patent Application no. PCT/U507/86188; WO 07/047,207; WO07/056,021; WO07/056,220; WO07/056,228; International Patent Application no. PCT/US08/62310; International Patent Application no. PCT/US08/062,793; International Patent Application no. PCT/US08/62580; International Patent Application no. PCT/US2008/052960; International Patent Application no. PCT/US08/81190; International Patent Application no. PCT/US08/76225; each of which is herein incorporated by reference in its entirety.

In one aspect, the FLAP inhibitor is selected from: MK886 (also known as 3-[3-tert-butylsulfanyl-1-(4-chloro-benzyl)-5-isopropyl-1H-indol-2-yl]-2,2-dimethyl-propionic acid); MK591 (also known as 3-[3-tert-butylsulfanyl-1-(4-chloro-benzyl)-5-(quinolin-2-ylmethoxy)-1H-indol-2-yl]-2,2-dimethyl-propionic acid); DG031 (also known as BAY X1005; cyclopentyl-[4-(quinolin-2-ylmethoxy)-phenyl]-acetic acid) Compound (3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid; prepared as outlined in U.S. patent application Ser. No. 11/553,946) Compound B (3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid; prepared as outlined in U.S. patent application Ser. No. 11/553,946); Compound C (3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid; prepared as outlined in WO 07/056,220); Compound D (3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid; see Compound 2-1 of U.S. patent application Ser. No. 11/553,946); Compound E (3-[3-tert-Butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid; see COMPOUND 2-10 of U.S. patent application Ser. No. 11/553,946); Compound F (3-[3-tert-Butylsulfanyl-1-[4-(5-fluoro-pyridin-2-yl)-benzyl]-5-(quinolin-2-ylmethoxy)-1H-indol-2-yl]-2,2-dimethyl-propionic acid; see Compound 2-19 of U.S. patent application Ser. No. 11/553,946); Compound G (2-[3-tert-Butylsulfanyl-1-[4-(5-methoxy-pyrimidin-2-yl)-benzyl]-5-(5-methyl-pyridin-2-ylmethoxy)-1H-indol-2-ylmethyl]-2-ethyl-butyric acid see Compound 4-3 of U.S. patent application Ser. No. 11/744,555); Compound H (3-[3-tert-Butylsulfanyl-1-[4-(6-methoxy-pyridin-3-yl)-benzyl]-5-(5-methyl-pyridin-2-ylmethoxy)-1H-indol-2-yl]-2,2-dimethyl-propionic acid; see Compound 2-7 of U.S. patent application Ser. No. 11/553,946); Compound I (3-[5-((S)-1-Acetyl-pyrrolidin-2-ylmethoxy)-3-tert-butylsulfanyl-1-(4-chloro-benzyl)-1H-indol-2-yl]-2,2-dimethyl-propionic acid; see Compound 1-2 of WO 07/056,220); Compound J (3-[3-tert-butylsulfanyl-1-[4-(5-fluoro-pyridin-2-yl)-benzyl]-5-(pyridin-2-ylmethoxy)-1H-indol-2-yl]-2,2-dimethyl-propionic acid; see Compound 2-201 of U.S. patent application no. 11/553,946); 3-{5-((S)-1-Acetyl-2,3-dihydro-1H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid (Compound K).

In another aspect, the FLAP inhibitor is selected from compounds described in U.S. Pat. Nos. 4,929,626; 4,970,215; 5,081,138; 5,095,031; 5,204,344; 5,126,354; 5,221,678; 5,229,516; 5,272,145; 5,283,252; 5,288,743; 5,292,769; 5,304,563; 5,399,699; 5,459,150; 5,512,581; 5,597,833; 5,668,146; 5,668,150; 5,691,351; 5,714,488; 5,783,586; 5,795,900; and 5,843,968, each of which is herein incorporated by reference for the disclosure of such FLAP inhibitors).

### Further Forms of FLAP Inhibitor Compounds

In some embodiments, the therapeutic agent(s) (e.g. FLAP inhibitor compound and/or second therapeutic agent) is present in the pharmaceutical composition as a pharmaceutically acceptable salt. In some embodiments, pharmaceutically acceptable salts are obtained by reacting a FLAP inhibitor compound with acids. In some other embodiments, pharmaceutically acceptable salts are obtained by reacting a FLAP inhibitor compound with a base. In other embodiments, the therapeutic agents are used as free-acid or free-base form in the manufacture of the pharmaceutical compositions described herein. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, FLAP inhibitor compounds described herein are reacted with an organic base, such as, but not limited to, ethanolamine, diethanolamine, triethanolamine, tromethamine, N-methylglucamine, dicyclohexylamine, tris(hydroxymethyl)methylamine. In other cases, FLAP inhibitor compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like In some embodiments, the FLAP inhibitor compounds described herein are used as the sodium salt.

In some embodiments, the FLAP inhibitor compounds described herein include solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol.

In some embodiments, the FLAP inhibitor compounds described herein possess one or more stereocenters and each center exists independently in either the R or S configuration. The compounds presented herein include all diastereomeric, enantiomeric, and epimeric forms as well as the appropriate mixtures thereof.

In some embodiments, sites on FLAP inhibitor compounds disclosed herein are susceptible to various metabolic reactions Therefore incorporation of appropriate substituents at the places of metabolic reactions will reduce, minimize or eliminate the metabolic pathways. In specific embodiments, the appropriate substituent to decrease or eliminate the susceptibility of the aromatic ring to metabolic reactions is, by way of example only, a halogen, deuterium or an alkyl group.

In some embodiments, FLAP inhibitor compounds described herein are labeled isotopically (e.g. with a radioisotope) or by another other means, including, but not limited to, the use of chromophores or fluorescent moieties, bioluminescent labels, or chemiluminescent labels. In some embodiments, FLAP inhibitor compounds described herein are isotopically-labeled, which are identical to those recited in the various formulae and structures presented herein, but for the fact that one or more atoms are replaced by an atom having an atomic mass or mass number different from the atomic mass or mass number usually found in nature. In some embodiments, one or more hydrogen atoms are replaced with deuterium. In some embodiments, metabolic sites on the compounds described herein are deuterated. In some embodiments, substitution with deuterium affords certain therapeutic advantages resulting from greater metabolic stability, such as, for example, increased in vivo half-life or reduced dosage requirements.

Throughout the specification, groups and substituents thereof can be chosen by one skilled in the field to provide stable moieties and compounds.

### Dermatological Diseases, Disorders or Conditions

Described herein, in certain embodiments, are topical formulations for treating a dermatological disease, disorder or condition (e.g., dermatoses). As used herein, a dermatological disease, disorder or condition includes any abnormal state of the epidermis, dermis, and/or subcutaneous tissues. In certain instances, a dermatological disease, disorder or condition is caused by an immune disease, disorder or condition, (e.g. an autoimmune disease, disorder or condition); a proliferative disease or condition; contact with an allergen and/or an irritant; a mast cell disease or condition, scarring, a burn, inflammatory disease or condition, or combinations thereof. Dermatological diseases, disorders or conditions include, but are not limited to, a chronic blistering (bullous) disorder, psoriasis, dermatitis (e.g., contact or atopic), eczema, urticaria, rosacea, scarring (i.e. the formation of a scar (e.g., a keloid scar or a hypertrophic scar)), a first degree burn, a second degree burn, a third degree burn, a fourth degree burn and/or melanoma.

In some embodiments, a topical formulation disclosed herein is administered before or after contact with an allergen and/or irritant. In some embodiments, a topical formulation disclosed herein is administered before or after a physical trauma (e.g., surgery).

In some embodiments, treating or preventing any of the diseases, disorders or conditions described herein reduces the severity of or prevents the occurence of at least one symptom of the disease, disorder or condition. In some embodiments, dermatological diseases, disorders or conditions are accompanied by inflammation of the upper layers of the skin. In some embodiments, inflammation of the upper layers of the skin causes itching, blisters, redness, swelling, oozing, scabbing, and scaling. In some embodiments, inflammation of the upper layers of the skin results in a rash, blisters, pimples, open sores, oozing, crusting, and scaling.

### Bullous Disease, Disorder or Condition

In some embodiments, a topical formulation disclosed herein is administered to treat a dermatological disease, disorder or condition, wherein the dermatological disease, disorder or condition is a bullous disease, disorder or condition. In certain instances, a bullous disease, disorder or condition is characterized by the formation of blisters (i.e., the accumulation of fluid between cells in the upper layers of the skin). In certain instances, bullous diseases, disorders or conditions are immune diseases, disorders or conditions. In certain instances, leukotrienes and/or cytokines mediate the formation of blisters (e.g., induce the exudation of plasma from capillaries to the upper layers of the skin). In certain instances, inhibiting FLAP activity reduces the concentration of leukotrienes associated with bullous disorders, and, further, treats bullous disorders. In certain instances, inhibiting FLAP activity reduces the concentration of cytokines associated with bullous diseases, disorders or conditions, and, further, treats bullous diseases, disorders or conditions. Bullous diseases, disorders or conditions include, but are not limited to, bullous pemphigoid, pemphigus vulgaris, pemphigus vegetans, pemphigus fallacious, paraneoplastic pemphigus, mucous membrane pemphigoid, linear IgA bullous disease, dermatitis herpeti-formis, and epidermolysis bullosa acquisita.

### Melanoma

In some embodiments, a topical formulation disclosed herein is administered to treat a dermatological disease, disorder or condition, wherein the dermatological disease, disorder or condition is melanoma. In one aspect, melanoma is a proliferative disorder of melanocytes. In certain instances, leukotrienes stimulate the growth of melanocytes. Further, in certain instances, inflammation facilitates the growth of melanoma. In one aspect, leukotrienes and/or cytokines mediate inflammation associated with melanoma. In certain instances, inhibiting FLAP activity reduces the concentration of leukotrienes and/or cytokines associated with melanoma, and slows and/or inhibits the growth of melanocytes associated with melanoma. In certain instances, inhibiting FLAP activity reduces inflammation associated with melanoma. In certain instances, inhibiting FLAP treats melanoma.

### Acne

In some embodiments, a topical formulation disclosed herein is administered to treat a dermatological disease, disorder or condition, wherein the dermatological disease, disorder or condition is acne. In one aspect, acne is caused by over-production of leukotrienes. In certain instances, acne is caused by sebum lipids (i.e., lipids produced by sebaceous glands). In certain instances, LTB₄ regulates production of sebum lipids. In certain instances, inhibiting FLAP activity reduces the concentration of LTB₄ and/or cytokines associated with acne. In certain instances, inhibiting FLAP activity reduces the concentration of leukotrienes and/or cytokines associated with acne. In certain instances, inhibiting FLAP activity reduces the concentration of sebum lipids associated with acne. In certain instances, inhibiting FLAP treats acne.

### Psoriasis

In some embodiments, a topical formulation disclosed herein is administered to treat a dermatological disease, disorder or condition, wherein the dermatological disease, disorder or condition is psoriasis. In certain instances, the symptoms of psoriasis result from the exudation of plasma from vessels and capillaries into the epidermis, dermis, and/or subcutaneous tissues. In certain instances, leukotrienes and/or cytokines cause the exudation of plasma from vessels and capillaries associated with psoriasis. In certain instances, inhibiting FLAP activity reduces the concentration of leukotrienes and/or cytokines associated with psoriasis. In certain instances, inhibiting FLAP activity reduces exudation of plasma from vessels and capillaries associated with psoriasis. In certain instances, inhibiting FLAP treats psoriasis.

### Dermatitis

In some embodiments, a topical formulation disclosed herein is administered to treat a dermatological disease, disorder or condition, wherein the dermatological disease, disorder or condition is dermatitis. As used herein, dermatitis means an inflammatory condition of the skin. In one aspect, the dermatitis is chronic or acute. In certain instances, dermatitis is acute and results from contact with an offending agent (e.g., uruishol). In certain instances, dermatitis is chronic and results from hypersensitivity.

Types of dermatitis include, but are not limited to: spongiotic dermatitis (irritant dermatitis, seborrheic dermatitis, atopic dermatitis, allergic contact dermatitis, thermal induced dermatitis, and drug induced dermatitis); allergic contact dermatitis (contact dermatitis can be due to external compounds, preservatives, fragrances, or plants); seborrhoeic dermatitis (seborrhoeic dermatitis is also known as dandruff); dyshidrotic dermatitis (also known as Pompholyx); vesicular or bullous dermatitis (can be caused by drug reaction, or auto immune diseases; examples includes Steven Johnson Syndrome, bullous erythema multiforme, bullous pemphigoid, and pemphigus vulgaris).

In certain instances, the symptoms of dermatitis (e.g., chronic or acute) result from a disorder of an immune system. In certain instances, the symptoms of dermatitis (e.g., chronic or acute) result from the exudation of plasma from vessels and capillaries into the epidermis, dermis, and/or subcutaneous tissues. In certain instances, leukotrienes and/or cytokines cause the exudation of plasma from vessels and capillaries. In certain instances, inhibiting FLAP activity reduces the concentration of leukotrienes and/or cytokines associated with dermatitis. In certain instances, inhibiting FLAP activity reduces exudation of plasma from vessels and capillaries associated with dermatitis. In certain instances, inhibiting FLAP treats dermatitis.

In some embodiments, dermatitis is atopic dermatitis or allergic dermatitis. In some embodiments, dermatitis is atopic dermatitis. In some embodiments, dermatitis is allergic dermatitis.

### Eczema

In some embodiments, a topical formulation disclosed herein is administered to treat a dermatological disease, disorder or condition, wherein the dermatological disease, disorder or condition is eczema. As used herein, eczema is a chronic inflammatory state of the skin. In certain instances, the symptoms of eczema result from the exudation of plasma from vessels and capillaries into the epidermis, dermis, and/or subcutaneous tissues. In certain instances, leukotrienes and/or cytokines cause the exudation of plasma from vessels and capillaries. In certain instances, inhibiting FLAP activity reduces the concentration of leukotrienes associated with eczema. In certain instances, inhibiting FLAP activity reduces the concentration of cytokines associated with eczema. In certain instances, inhibiting FLAP activity reduces exudation of plasma from vessels and capillaries associated with eczema. In certain instances, inhibiting FLAP treats eczema.

### Urticaria

In some embodiments, a topical formulation disclosed herein is administered to treat a dermatological disease, disorder or condition, wherein the dermatological disease, disorder or condition is urticaria. In certain instances, urticaria results from hypersensitivity or another immune disorder. In certain instances, the symptoms of urticaria result from the exudation of plasma from vessels and capillaries into the epidermis, dermis, and/or subcutaneous tissues. In certain instances, leukotrienes cause the exudation of plasma from vessels and capillaries. In certain instances, cytokines cause the inflammation and/or hypersensitivity associated with urticaria. In certain instances, inhibiting FLAP activity reduces the concentration of leukotrienes and/or cytokines associated with urticaria. In certain instances, inhibiting FLAP reduces exudation of plasma from vessels and capillaries associated with urticaria. In certain instances, inhibiting FLAP treats urticaria.

### Rosacea

In some embodiments, a topical formulation disclosed herein is administered to treat a dermatological disease, disorder or condition, wherein the dermatological disease, disorder or condition is rosacea. As used herein, rosacea refers to any of erythematotelangiectatic rosacea (ETR), Papulopustular rosacea, and/or Phymatous rosacea. In certain instances, inhibiting FLAP activity treats rosacea. In certain instances, reducing the concentration of leukotrienes and/or cytokines treats rosacea.

### Skin Ulcers

In some embodiments, a topical formulation disclosed herein is administered to treat a dermatological disease, disorder or condition, wherein the dermatological disease, disorder or condition is skin ulcers. As used herein, an ulcer is a disorder of the skin characterized by degradation of the epidermis and often portions of the dermis and even subcutaneous fat. In certain instances, ulcers are areas of necrotic tissue. In certain instances, ulcers result from immune system dysfunction. In certain instances, ulcers result from immune system dysfunction such as, but not limited to, the improper functioning of neutrophils. In certain instances, leukotrienes are chemotactic agents for neutrophils. In certain instances, cytokines cause the inflammation and/or hypersensitivity associated with skin ulcers. In certain instances, inhibiting FLAP activity reduces the concentration of leukotrienes associated with skin ulcers. In certain instances, inhibiting FLAP activity reduces the chemotaxis of neutrophils associated with skin ulcers. In certain instances, inhibiting FLAP activity reduces the concentration of cytokines associated with skin ulcers. In certain instances, inhibiting FLAP treats skin ulcers.

### Scarring

In some embodiments, a topical formulation disclosed herein is administered to treat a dermatological disease, disorder or condition, wherein the dermatological disease, disorder or condition is scarring. As used herein, scarring refers to the formation of a scar. In one aspect, the scar is a hypertrophic scar, or keloid scar, or a scar resulting from acne. In certain instances, a scar is an area of fibrous tissue that results from inflammation (e.g., the overproduction of cytokines and/or collagen). In certain instances a scar is a result of an infection (e.g. acne). In certain instances, fibroblasts migrate and deposit excess collagen at the wound site, resulting in a scar. In certain instances, leukotrienes act as chemotactic agents for fibroblasts and modulate the fibroblasts' activity. In certain instances, inhibiting the activity of FLAP inhibits the activity and/or migration of fibroblasts associated with scarring. In certain instances, inhibiting FLAP activity reduces the concentration of leukotrienes associated with scarring. In certain instances, inhibiting FLAP inhibits the activity of and/or migration of fibroblasts associated with scarring. In certain instances, cytokines modulate the inflammation associated with scarring. In certain instances, inhibiting FLAP reduces or inhibits the concentration of cytokines associated with scarring. In certain instances, inhibiting FLAP activity treats scarring.

### Burns

In some embodiments, a topical formulation disclosed herein is administered to treat a dermatological disease, disorder or condition, wherein the dermatological disease, disorder or condition is a burn. As used herein, a burn refers to an injury to or the destruction of skin caused by heat, cold, electricity, chemicals, light (e.g. a sunburn caused by UV exposure), radiation, or friction. In one aspect, the burn is a first degree burn, a second degree burn, a third degree burn, or a fourth degree burn. In certain instances, a burn results in the formation of a scar. In certain instances, a burn results in inflammation. In certain instances, inhibiting the activity of FLAP inhibits the activity and/or migration of leukocytes associated with scarring and/or inflammation. In certain instances, inhibiting FLAP activity reduces the concentration of leukotrienes associated with scarring and/or inflammation. In certain instances, inhibiting FLAP activity reduces the concentration of cytokines associated with scarring and/or inflammation. In certain instances, inhibiting FLAP activity treats scarring and/or inflammation.

### Itch

In some embodiments, a topical formulation disclosed herein is administered to treat or prevent itching in a mammal. In some embodiments, the itching is a symptom of any of the diseases, disorders or conditions disclosed herein. In some embodiments, the itching is a result of contact with an irritant, allergen, or combination thereof. In some embodiments, the topical formulations disclosed herein reduce itching that is associated with contact with an irritant, allergen, or combination thereof. In some embodiments, the topical formulations disclosed herein reduce itching that is associated with dermatitis, psoriasis or uticaria.

### Certain Terminology

The terms “individual,” “patient,” or “subject” are used interchangeably. As used herein, they mean any mammal. In one aspect, the mammal is a human.

The terms “treat,” “treating” or “treatment,” and other grammatical equivalents as used herein, include alleviating, abating, inhibiting, reducing, ameliorating, delaying the onset of, arresting the progression of, and/or inducing the regression of a disorder and/or the symptoms of a disorder. The terms also include prophylactic treatment of a disorder. The terms further include achieving any therapeutic benefit. Therapeutic benefit means the eradication or amelioration of the underlying disorder being treated, and/or the eradication or amelioration of one or more of the physiological symptoms associated with the underlying disorder such that an improvement is observed in the individual.

The terms “prevent,” “preventing” or “prevention,” and other grammatical equivalents as used herein include inhibiting (arresting or stopping) the development of a disorder, and/or inhibiting (arresting or stopping) the further progression of a disorder. These terms are intended to include prophylaxis. For prophylactic benefit, the compositions are administered to an individual at risk of developing a particular disorder, or to an individual reporting one or more of the physiological symptoms of a disease, or to an individual at risk of reoccurrence of the disease.

The terms “effective amount” or “therapeutically effective amount” as used herein, refer to an amount of an agent (e.g. FLAP inhibitor compound) being administered which achieve a desired result, e.g., to relieve to some extent one or more symptoms of a disease, disorder or condition being treated. In certain instances, the result is a reduction and/or alleviation of at least one sign, symptom, or cause of a disease, or any other desired alteration of a biological system.

### Topical Formulations

In some embodiments, a topical formulation disclosed herein facilitates the delivery of a FLAP inhibitor compound to the skin for a local effect (i.e., an effect that is limited to the skin). In certain instances, local administration of a FLAP inhibitor compound reduces or eliminates side-effects that are associated with systemic administration of a FLAP inhibitor compound.

Topical formulations include, but are not limited to, ointments, creams, lotions, solutions, pastes, gels, sticks, liposomes, nanoparticles, patches, bandages and wound dressings.

In some embodiments, any dermal formulation described herein comprises between about 0.1 to about 50%, between about 0.1 to about 25%, between about 0.1 to about 10%, between about 0.1 to about 5%, or between about 0.1 to about 1% of a FLAP inhibitor compound by weight of the formulation.

### Creams and Lotions

Disclosed herein, in certain embodiments, is a topical formulation of a FLAP inhibitor compound wherein the topical formulation is in the form of a cream. In certain instances, creams are semisolid (e.g., soft solid or thick liquid) formulations that include a FLAP inhibitor compound dispersed in an oil-in-water emulsion or a water-in-oil emulsion. Disclosed herein, in certain embodiments, is a topical formulation of a FLAP inhibitor compound wherein the topical formulation is in the form of a lotion. In certain instances, lotions are fluid emulsions (e.g., oil-in-water emulsions or a water-in-oil emulsions). In some embodiments, the hydrophobic component of a lotion and/or cream is derived from an animal (e.g., lanolin, cod liver oil, and ambergris), plant (e.g., safflower oil, castor oil, coconut oil, cottonseed oil, menhaden oil, palm kernel oil, palm oil, peanut oil, soybean oil, rapeseed oil, linseed oil, rice bran oil, pine oil, sesame oil, or sunflower seed oil), or petroleum (e.g., mineral oil, or petroleum jelly).

In certain instances, lotions and creams have a “drying” effect on dermatological disorders (e.g., some or all fluid exuded from the disorder is miscible in the ointment) and are thus useful for dermatological disorders characterized by the exudation of fluids.

### Ointments

Disclosed herein, in certain embodiments, is a topical formulation of a FLAP inhibitor compound wherein the topical formulation is in the form of an ointment. In certain instances, ointments are semisolid preparations that soften or melt at body temperature. In certain instances, ointments re-hydrate the skin and are thus useful for dermatological diseases, disorders or conditions characterized by loss of moisture.

### Pastes

Disclosed herein, in certain embodiments, is a topical formulation of a FLAP inhibitor compound wherein the topical formulation is in the form of a paste. In certain instances, pastes contain at least 20% solids. In certain instances, pastes are ointments that do not flow at body temperature. In certain instances, pastes re-hydrate the skin and are thus useful for dermatological diseases, disorders or conditions characterized by loss of moisture. In certain instances, pastes serve as protective coatings over areas to which they are applied.

### Gels

Disclosed herein, in certain embodiments, is a topical formulation of a FLAP inhibitor compound wherein the topical formulation is in the form of a gel. In certain instances, gels are semisolid (or semi-rigid) systems consisting of dispersions of large organic molecules dispersed in a liquid. In certain instances, gels are water-soluble and are removed using warm water or saline. In certain instances, gels re-hydrate the skin and are thus useful for dermatological diseases, disorders or conditions characterized by loss of moisture.

Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums, (e.g., tragacanth). In some embodiments, single-phase gels are generally aqueous, but will also be made using alcohols and oils. Two-phase gels consist of a network of small discrete particles.

Gels can also be classified as being hydrophobic or hydrophilic. In certain embodiments, the base of a hydrophobic gel consists of a liquid paraffin with polyethylene or fatty oils gelled with colloidal silica, or aluminum or zinc soaps. In contrast, the base of hydrophobic gels usually consists of water, glycerol, or propylene glycol gelled with a suitable gelling agent (e.g., tragacanth, starch, cellulose derivatives, carboxyvinylpolymers, and magnesium-aluminum silicates).

### Sticks

Disclosed herein, in certain embodiments, is a topical formulation of a FLAP inhibitor compound wherein the topical formulation is in the form of a stick. In certain instances, sticks are solid dosage forms that melt at body temperature. In some embodiments, a stick comprises a wax, a polymer, a resin, dry solids fused into a firm mass, and/or fused crystals. In some embodiments, a topical formulation of a FLAP inhibitor compound is in the form of a styptic pencil (i.e., a stick prepared by (1) heating crystals until they lose their water of crystallization and become molten, and (2) pouring the molten crystals into molds and allowing them to harden). In some embodiments, a topical formulation of a FLAP inhibitor compound is in the form of stick wherein the stick comprises a wax (e.g., the wax is melted and poured into appropriate molds in which they solidify in stick form).

In some embodiments, a topical formulation of a FLAP inhibitor compound is in the form of stick wherein the stick comprises a melting base (i.e., a base that softens at body temperature). Examples of melting bases include, but are not limited to, waxes, oils, polymers and gels. In some embodiments, a topical formulation of a FLAP inhibitor compound is in the form of stick wherein the stick comprises a moisten base (i.e., a base that is activated by the addition of moisture).

### Patches

Disclosed herein, in certain embodiments, is a topical formulation of a FLAP inhibitor compound wherein the topical formulation is administered via a patch. In some embodiments, a topical formulation disclosed herein is dissolved and/or dispersed in a polymer or an adhesive. In some embodiments, a patch disclosed herein is constructed for continuous, pulsatile, or on demand delivery of a FLAP inhibitor compound.

In some instances, the topical formulations described herein comprise pressure sensitive adhesives (e.g., polyalkyloxazoline polymers) and allow for application of an adhesive film to an affected area of skin.

### Wound Dressings

Disclosed herein, in certain embodiments, is a topical formulation of a FLAP inhibitor compound wherein the topical formulation is administered with (or via) a wound dressing. Wound dressings include, but are not limited to gauzes, transparent film dressings, hydrogels, polyurethane foam dressings, hydrocolloids and alginates. In certain instances, wound dressings (1) maintain moisture in the wound, (2) are semipermeable, (3) are semiocclusive, (4) allow for autolytic debridement, (5) protect from external contaminants, (6) absorb exuded fluids, and/or (7) allow for wound visualization.

### Dermal Paints

In some embodiments, the formulations and compositions disclosed herein are administered as a dermal paint. As used herein, paints (also known as film formers) are solutions comprised of a solvent, a monomer or polymer, an active agent, and optionally one or more pharmaceutically-acceptable excipients. After application to a tissue, the solvent evaporates leaving behind a thin coating comprised of the monomers or polymers, and the active agent. The coating protects active agents and maintains them in an immobilized state at the site of application. This decreases the amount of active agent which may be lost and correspondingly increases the amount delivered to the affected area of the skin of an individual. By way of non-limiting example, paints include collodions (e.g. Flexible Collodion, USP), and solutions comprising saccharide siloxane copolymers and a cross-linking agent. Collodions are ethyl ether/ethanol solutions containing pyroxylin (a nitrocellulose). After application, the ethyl ether/ethanol solution evaporates leaving behind a thin film of pyroxylin. In solutions comprising saccharide siloxane copolymers, the saccharide siloxane copolymers form the coating after evaporation of the solvent initiates the cross-linking of the saccharide siloxane copolymers.

### Dermatological Excipients

Disclosed herein, in certain embodiments, is a topical formulation of a FLAP inhibitor compound wherein the topical formulation comprises a penetration enhancer. Penetration enhancers include, but are not limited to, sodium lauryl sulfate, sodium laurate, polyoxyethylene-20-cetyl ether, laureth-9, sodium dodecylsulfate, dioctyl sodium sulfosuccinate, polyoxyethylene-9-lauryl ether (PLE), Tween 80, nonylphenoxypolyethylene (NP-POE), polysorbates, sodium glycocholate, sodium deoxycholate, sodium taurocholate, sodium taurodihydrofusidate, sodium glycodihydrofusidate, oleic acid, caprylic acid, mono- and di-glycerides, lauric acids, acylcholines, caprylic acids, acylcarnitines, sodium caprates, EDTA, citric acid, salicylates, DMSO, decylmethyl sulfoxide, ethanol, isopropanol, propylene glycol, polyethylene glycol, glycerol, propanediol, and diethylene glycol monoethyl ether.

Disclosed herein, in certain embodiments, is a topical formulation of a FLAP inhibitor compound wherein the topical formulation comprises a gelling (or thickening) agent. In some embodiments, a topical formulation disclosed herein further comprises from about 0.1% to about 5%, more preferably from about 0.1% to about 3%, and most preferably from about 0.25% to about 2%, of a gelling agent. In certain embodiments, the viscosity of a topical formulation disclosed herein is in the range from about 100 to about 500,000 cP, about 100 cP to about 1,000 cP, about 500 cP to about 1500 cP, about 1000 cP to about 3000 cP, about 2000 cP to about 8,000 cP, about 4,000 cP to about 10,000 cP, about 10,000 cP to about 50,000 cP.

Suitable gelling agents for use in preparation of the gel topical formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, acacia (gum arabic), agar, aluminum magnesium silicate, sodium alginate, sodium stearate, bladderwrack, bentonite, carbomer, carrageenan, carbopol, xanthan, cellulose, microcrystalline cellulose (MCC), ceratonia, chondrus, dextrose, furcellaran, gelatin, ghatti gum, guar gum, hectorite, lactose, sucrose, maltodextrin, mannitol, sorbitol, honey, maize starch, wheat starch, rice starch, potato starch, gelatin, sterculia gum, polyethylene glycol (e.g. PEG 200-4500), gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), or combinations thereof.

Suitable agents for use in formulations that are applied as liquids and gel upon application to the skin into a film include but are not limited to polymers composed of polyoxypropylene and polyoxyethylene that are known to form thermoreversible gels when incorporated into aqueous solutions. These polymers have the ability to change from the liquid state to the gel state at temperatures close to body temperature, therefore allowing useful formulations that are applied as gels and/or films to the affected area. Examples of polymers that gel at body temperature and are used in gels and/or films described herein include and are not limited to poloxamers (e.g., PLURONICS F68®, F88®, F108®, and F127®, which are block copolymers of ethylene oxide and propylene oxide). The liquid state-to-gel state phase transition is dependent on the polymer concentration and the ingredients in the solution.

Disclosed herein, in certain embodiments, is a topical formulation of a FLAP inhibitor compound wherein the topical formulation comprises an emollient. Emollients include, but are not limited to, castor oil esters, cocoa butter esters, safflower oil esters, cottonseed oil esters, corn oil esters, olive oil esters, cod liver oil esters, almond oil esters, avocado oil esters, palm oil esters, sesame oil esters, squalene esters, kikui oil esters, soybean oil esters, acetylated monoglycerides, ethoxylated glyceryl monostearate, hexyl laurate, isohexyl laurate, isohexyl palmitate, isopropyl palmitate, methyl palmitate, decyloleate, isodecyl oleate, hexadecyl stearate decyl stearate, isopropyl isostearate, methyl isostearate, diisopropyl adipate, diisohexyl adipate, dihexyldecyl adipate, diisopropyl sebacate, lauryl lactate, myristyl lactate, and cetyl lactate, oleyl myristate, oleyl stearate, and oleyl oleate, pelargonic acid, lauric acid, myristic acid, palmitic acid, stearic acid, isostearic acid, hydroxystearic acid, oleic acid, linoleic acid, ricinoleic acid, arachidic acid, behenic acid, erucic acid, lauryl alcohol, myristyl alcohol, cetyl alcohol, hexadecyl alcohol, stearyl alcohol, isostearyl alcohol, hydroxystearyl alcohol, oleyl alcohol, ricinoleyl alcohol, behenyl alcohol, erucyl alcohol, 2-octyl dodecanyl alcohol, lanolin and lanolin derivatives, beeswax, spermaceti, myristyl myristate, stearyl stearate, carnauba wax, candelilla wax, lecithin, and cholesterol.

Disclosed herein, in certain embodiments, is a topical formulation of a FLAP inhibitor compound wherein the topical formulation comprises abrasives, absorbents, anticaking agents, astringents, essential oils, fragrances, skin-conditioning agents, skin healing agents, skin protectants (e.g., sunscreens, or ultraviolet light absorbers or scattering agents), skin soothing agents, preservatives or combinations thereof.

Pharmaceutical topical formulations disclosed herein are formulated in any suitable manner. Any suitable technique, carrier, and/or excipient is contemplated for use with the FLAP inhibitors disclosed herein. For a summary of pharmaceutical topical formulations described herein see Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Eighth Ed. (Lippincott Williams & Wilkins 2004), Muller, R. H. et al. Advanced Drug Delivery Reviews 59 (2007) 522-530, which are herein incorporated by reference for such disclosures.

### Dosing

Disclosed herein, in certain embodiments, is a topical formulation of a FLAP inhibitor compound wherein the topical formulation administered for prophylactic and/or therapeutic treatments. In certain instances, amounts effective for this use will depend on the severity and course of the disease, disorder or condition, previous therapy, the individual's health status and response to the drugs, and the judgment of the treating physician. In some embodiments, the dose is about 0.001% by weight to about 10% by weight of the formulation.

In some embodiments, where a dermatological disorder does not improve, a topical formulation disclosed herein is administered chronically (i.e., for an extended period of time, including throughout the duration of the individual's life). In some embodiments, where a dermatological disorder does improve, a topical formulation disclosed herein is given continuously; alternatively, the dose of active agent being administered is temporarily reduced or temporarily suspended for a certain length of time (i.e., a “drug holiday”). In some embodiments, a drug holiday lasts between 2 days and 1 year, including all integers in between. In some embodiments, the dose reduction during a drug holiday is from about 10% to about 100%, including all integers in between.

In some embodiments, where a dermatological disorder does improve, a topical formulation disclosed herein is administered as a maintenance dose. In some embodiments, where a dermatological disorder does improve, a topical formulation disclosed herein is administered with reduced frequency or at a reduced dose.

In one embodiment, a topical formulation disclosed herein is formulated for controlled release of a FLAP inhibitor compound. In some embodiments, a FLAP inhibitor compound is released over a time period exceeding 15 minutes, or 30 minutes, or 1 hour, or 4 hours, or 6 hours, or 12 hours, or 18 hours, or 1 day, or 2 days, or 3 days, or 4 days, or 5 days, or 6 days, or 7 days, or 10 days, or 12 days, or 14 days, or 18 days, or 21 days, or 25 days, or 30 days, or 45 days, or 2 months or 3 months or 4 months or 5 months or 6 months or 9 months or 1 year.

### Combination Therapy

In one aspect, pharmaceutical compositions and methods disclosed herein include an additional therapeutic agent. In one aspect, the additional therapeutic agent is a therapeutic agent other than a FLAP inhibitor compound.

In one aspect, the topical formulations disclosed herein that include a FLAP inhibitor compound are co-administered with (either separately or in the same formulation) a therapeutic agent selected from: antibiotics (e.g., polymyxin B sulfate/bacitracin zinc, polymyxin B/neomycin/gramicidin, polymyxin B/trimetoprim, polymyxin B/bacitracin, fluoroquinolones (e.g., ciprofloxacin, moxifloxacin, ofloxacin, gatifloxacin, levofloxacin), aminoglycosides (e.g. tobramycin, azithromycin, gentamicin, erythromycin, bacitracin); anti-fungal agents (e.g., amphotericin B, itraconazole, fluconazole, voriconazole); steroid anti-inflammatory agents (e.g., fluorometholone acetate, prednisolone acetate, loteprednol etabonate, prednisolone sodium phosphate, prednisolone sodium, rimexolone, fluorometholone acetate); non-steroidal anti-inflammatory agents (e.g., nepafenac, ketorolac tromethamine, bromfenac, diclofenac sodium, ketorolac tromethamine, ketotifen fumarate); antihistamines (e.g., emedastine difumarate, olopatadine hydrochloride, epinastine HCl, Azelastine Hydrochloride, ketotifen fumarate); antivirals (e.g., acyclovir, vidarabine, trifluridine); alpha agonists (e.g., apraclonidine, brimonidine, bimatoprost); beta blockers (e.g., betaxolol hydrochloride, levobunolol hydrochloride, carteolol hydrochloride, metipranolol, timolol maleate, timolol hemihydrate); carbonic anhydrase inhibitors (e.g., brinzolamide, dorzolamide, acetazolamide); miotics (e.g., acetylcholine chloride, echothiophate); prostaglandins (e.g., travoprost, bimatoprost, latanoprost); anti-angiogenesis agents (e.g., pegaptanib sodium, ranibizumab, verteporfin); loteprednol etabonate, mast cell stabilizers (e.g., lodoxamide tromethamine, nedocromil sodium, cromolyn sodium, pemirolast potassium), cyclosporine, and DP2 antagonists.

In some embodiments, the topical pharmaceutical compositions disclosed herein comprising a FLAP inhibitor compound are co-administered with (either separately or in the same formulation) an antibiotic. Antibiotics include, but are not limited to polymyxin B sulfate/bacitracin zinc, polymyxin B/neomycin/gramicidin, polymyxin B/trimetoprim, polymyxin B/bacitracin, fluoroquinolones (e.g., ciprofloxacin, moxifloxacin, ofloxacin, gatifloxacin, levofloxacin), aminoglycosides (e.g. tobramycin, azithromycin, gentamicin, erythromycin, bacitracin).

In some embodiments, the topical pharmaceutical compositions disclosed herein comprising a FLAP inhibitor compound are co-administered with (either separately or in the same formulation) an anti-fungal agent. Anti-fungal agents include, but are not limited to amphotericin B, itraconazole, fluconazole, and voriconazole.

In some embodiments, the topical pharmaceutical compositions disclosed herein comprising a FLAP inhibitor compound are co-administered with (either separately or in the same formulation) a steroid anti-inflammatory agent. Steroid anti-inflammatory agents include but are not limited to, betamethasone, prednisone, aclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumethasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone/prednisolone, rimexolone, tixocortol, triamcinolone, and ulobetasol.

In some embodiments, the topical pharmaceutical compositions disclosed herein comprising a FLAP inhibitor compound are co-administered with (either separately or in the same formulation) a non-steroidal anti-inflammatory agent (NSAID). NSAIDs include, but are not limited to, nepafenac, ketorolac, bromfenac, diclofenac, ketorolac, ketotifen.

In some embodiments, the topical pharmaceutical compositions disclosed herein comprising a FLAP inhibitor compound are co-administered with (either separately or in the same formulation) an antihistamine. In some embodiments, antihistamines include, but are not limited to, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine, and triprolidine. In some embodiments, antihistamines include, but are not limited to, emedastine, olopatadine, epinastine, azelastine, ketotifen.

In some embodiments, the topical pharmaceutical compositions disclosed herein comprising a FLAP inhibitor compound are co-administered with (either separately or in the same formulation) an antiviral agent. Antiviral agents include, but are not limited to, acyclovir, vidarabine, trifluridine.

In some embodiments, the topical pharmaceutical compositions disclosed herein comprising a FLAP inhibitor compound are co-administered with (either separately or in the same formulation) and alpha agonist. Alpha agonists include, but are not limited to, apraclonidine, brimonidine, bimatoprost.

In some embodiments, the topical pharmaceutical compositions disclosed herein comprising a FLAP inhibitor compound are co-administered with (either separately or in the same formulation) a beta blocker. Beta blockers include, but are not limited to, betaxolol, levobunolol, carteolol, metipranolol, timolol.

In some embodiments, the topical pharmaceutical compositions disclosed herein comprising a FLAP inhibitor compound are co-administered with (either separately or in the same formulation) a carbonic anhydrase inhibitor. Carbonic anhydrase inhibitors include, but are not limited to, brinzolamide, dorzolamide, acetazolamide.

In some embodiments, the topical pharmaceutical compositions disclosed herein comprising a FLAP inhibitor compound are co-administered with (either separately or in the same formulation) a miotic. Miotics include, but are not limited to, acetylcholine chloride, echothiophate.

In some embodiments, the topical pharmaceutical compositions disclosed herein comprising a FLAP inhibitor compound are co-administered with (either separately or in the same formulation) a prostaglandin. Prostaglandins include, but are not limited to, travoprost, bimatoprost, latanoprost.

In some embodiments, the topical pharmaceutical compositions disclosed herein comprising a FLAP inhibitor compound are co-administered with (either separately or in the same formulation) an anti-angiogenesis agent. Anti-angiogenesis agents include, but are not limited to, pegaptanib sodium, ranibizumab, verteporfin.

In some embodiments, the topical pharmaceutical compositions disclosed herein comprising a FLAP inhibitor compound are co-administered with (either separately or in the same formulation) loteprednol etabonate.

In some embodiments, the topical pharmaceutical compositions disclosed herein comprising a FLAP inhibitor compound are co-administered with (either separately or in the same formulation) a mast cell stabilizer. Mast cell stabilizers include, but are not limited to, lodoxamide tromethamine, nedocromil sodium, cromolyn sodium, pemirolast potassium.

In some embodiments, the topical pharmaceutical compositions disclosed herein comprising a FLAP inhibitor compound are co-administered with (either separately or in the same formulation) cyclosporine.

In some embodiments, the topical pharmaceutical compositions disclosed herein comprising a FLAP inhibitor compound are co-administered with (either separately or in the same formulation) a DP₂ receptor antagonist. In one aspect, the additional therapeutic agent is a small molecule DP₂ receptor antagonist compound. In some embodiments, a DP₂ receptor antagonist is selected from compounds disclosed in International patent application no. PCT/US09/35174 (entitled Antagonists of Prostaglandin D₂ receptors); International patent application no. PCT/US08/82056 (entitled Antagonists of PGD₂ receptors); International patent application no. PCT/US08/82082 (entitled Antagonists of PGD₂ receptors); International patent application no. PCT/US0932495 (entitled N,N-disubstituted aminoalkylbiphenyl antagonists of prostaglandin D₂ receptors); International patent application no. PCT/US09/32499 (entitled “N,N-disubstituted aminoalkylbiphenyl antagonists of prostaglandin D₂ receptors”); International patent application no. PCT/US09/33961 (entitled “Cyclic diaryl ether compounds as antagonists of prostaglandin D₂ receptors”); International patent application no. PCT/US09/38291 (entitled “Aminoalkylphenyl antagonists of prostaglandin D₂ receptors”); International patent application no. PCT/US09/49621 (entitled “Antagonists of prostaglandin D₂ receptors”); International patent application no. PCT/US09/49631 (entitled “Antagonists of prostaglandin D₂ receptors”); International patent application no. PCT/US09/58655 (entitled “Heteroaryl antagonists of prostaglandin D₂ receptors”); International patent application no. PCT/US09/58663 (entitled “Heteroaryl antagonists of prostaglandin D₂ receptors”); International patent application no. PCT/US09/44219 (entitled “Tricyclic antagonists of prostaglandin D₂ receptors”); International patent application no. PCT/US09/48327 (entitled “Cycloaklane[B]indole antagonists of prostaglandin D₂ receptors”); International patent application no. PCT/US09/59256 (entitled “Heteroaryl antagonists of prostaglandin D₂ receptors”); International patent application no. PCT/US09/59891 (entitled “Heteroalkyl biphenyl antagonists of prostaglandin D₂ receptors”); International patent application no. PCT/US09/64630 (entitled “Heterocyclic antagonists of prostaglandin D₂ receptors”); International patent application no. PCT/US09/63439 (entitled “Cycloaklane[B]azaindole antagonists of prostaglandin D2 receptors”); International patent application no. PCT/US09/63438 (entitled “Cycloaklane[B]azaindole antagonists of prostaglandin D2 receptors”); U.S. provisional application No. 61/147,437 (entitled “Indolozine compounds as prostaglandin D₂ receptor antagonists”); or pharmaceutically acceptable salts or N-oxides thereof.

In some embodiments, the DP₂ receptor antagonist is selected from AMG 009, AMG 853, Compound 14 of WO 09/085,177, AZD1981, ODC9101 (OC459), OC499, OC1768, OC2125, OC2184, QAV680, MLN6095, ACT-129968, ADC3680, SAR398171, S555739, AP768, [2′-(3-Benzyl-1-ethyl-ureidomethyl)-6-methoxy-4′-trifluoromethyl-biphenyl-3-yl]-acetic acid, {3-[2-tert-Butylsulfanylmethyl-4-(2,2-dimethyl-propionylamino)-phenoxy]-4-methoxy-phenyl}-acetic acid, TM30642, TM30643, TM30089, TM27632, and TM3170, {2′-[(N-cyclopropanecarbonyl-N-ethyl-amino)-methyl]-6-methoxy-4′-trifluoromethyl-biphenyl-3-yl}-acetic acid, [2′-[(N-cyclopropanecarbonyl-N-ethyl-amino)-methyl]-4′-(6-ethoxy-pyridin-3-yl)-6-methoxy-biphenyl-3-yl]-acetic acid, (5-{2-[(N-benzyloxycarbonyl-N-ethyl-amino)-methyl]-4-trifluoromethyl-phenyl}-pyridin-3-yl)-acetic acid, and {8-[(4-fluoro-benzenesulfonyl)-methyl-amino]-6,7,8,9-tetrahydro-pyrido[3,2-b]indol-5-yl}-acetic acid.

In some embodiments, the FLAP inhibitor and the additional therapeutic agent are in the same pharmaceutical composition. In some embodiments, the FLAP inhibitor and the additional therapeutic agent are in separate pharmaceutical compositions. In some embodiments, the FLAP inhibitor and the additional therapeutic agent are administered at the same time. In some embodiments, the FLAP inhibitor and the additional therapeutic agent are administered at different times.

### EXAMPLES

The following examples are illustrative and non-limiting to the scope of the formulations and methods described herein.

### Example 1

### Topical Formulation of a FLAP Inhibitor

In one aspect, a topical formulation of a FLAP inhibitor compound is prepared by mixing a FLAP inhibitor compound with propylene glycol, transcutol and water. In one aspect, the topical formulation includes a FLAP inhibitor compound (10 mg/mL) in a solution of 75% propylene glycol, 15% transcutol, 10% water.

Using this formulation, the following FLAP inhibitor compounds are all soluble at 10 mg/mL: Compound A (Na salt), Compound B (Na salt), Compound C (acid and Na salt), Compound F (Na salt), Compound G (Na salt), Compound I (acid), and Compound J (Na salt).

### Example 2

### Lotion Formulation of Compound A

Compound A is formulated as follows:

| Ingredient | Amount | |
|---|---|---|
| Compound A | 15 | g |
| Menthol | 3750 | mg |
| Polysorbate 80 | 3.75 | mL |
| Ethanol 95% | 375 | mL |
| Propylene glycol | 375 | mL |
| Purified water | qs 1500 | mL |

Compound A is mixed with menthol and polysorbate 80. Subsequently, propylene glycol and ethanol are added to the mixture. Finally, the volume of the mixture is brought up to 1500 mL with purified water.

### Example 3

### Lotion Formulation of Compound B

Compound B is formulated as follows:

| Ingredient | Amount | |
|---|---|---|
| Compound B | 90 | g |
| Ethanol 95% | 2250 | mL |
| Ethoxy diglycol | 300 | mL |
| Methylparaben | 22500 | mg |
| Propylparaben | 22.5 | mL |
| Propylene glycol | 2250 | mL |
| Purified water | qs 9000 | mL |

Compound B is mixed with the ethanol, until Compound B is dissolved. Subsequently, ethoxy diglycol is added. Further, methylparaben and propylparaben are mixed into the propylene glycol. The Compound B/ethanol mixture is then added to the propylene glycol/methylparaben/propylparaben mixture. Finally, the volume up is brought up to 9000 mL with purified water.

### Example 4

### Hydrogel Formulation of Compound C

Compound C is formulated as follows:

| Ingredient | Amount | |
|---|---|---|
| Compound C | 100 | g |
| Benzyl alcohol | 40 | mL |
| Glycerine | 400 | mL |
| Carbopol 934P | 40 | g |
| Trolamine | qs until a gel forms | |
| Purified water | qs 4000 | mL |

Compound C, benzyl alcohol, and glycerin are added to about 3200 mL of purified water. Carbopol is slowly added to the mixture. The volume is brought up to 4000 mL with purified water. Finally, trolamine is added dropwise until a gel is formed.

### Example 5

### Stick Formulation of Compound D

Compound D is formulated as follows:

| Ingredient | Amount | |
|---|---|---|
| Compound D | 50 | g |
| Beeswax | 340 | g |
| Cocoa Butter | 80 | g |
| Lanolin | 60 | g |
| Petroleum | 180 | g |
| Paraffin WaX | 100 | g |

Beeswax, cocoa butter, paraffin and lanolin are first melted. Compound D is then mixed with the petrolatum. The Petrolatum/Compound D mixture is added to melted formulation. The resultant mixture is stirred and poured into mold.

### Example 6

### Clinical Trial Evaluating Effect of a FLAP Inhibitor Compound on the Treatment and Prevention of Recurrence of Excised Keloids. Treatment at time of Surgery

A double blind, randomized, placebo controlled within trial study to evaluate the safety and efficacy of a compound administered topically following the excision of a keloid scar on the ear lobe. Each patient undergoes bilateral keloid scar excision and one ear lobe is treated with compound while the other ear lobe is treated with placebo such that each patient will act as their own control.

Ten to twelve subjects aged 18-65 years are to participate in the study. All subjects should have bilateral keloid scars suitable for surgical excision such that, following excision, the result will be a single wound on each ear lobe no greater than 2 cm long. The wound will be restricted to the skin, fat and fibrous tissue of the ear lobe.

No subject should have experienced keloid treatment with irradiation, cryosurgery, corticosteroids or other pharmacological agents within 12 weeks prior to the study. Subjects should not have a history of a bleeding disorder and should not have experienced or have on-going psoriasis or eczema or malignant skin tumors. Female subjects of child bearing potential must have a documented negative urine pregnancy test and must be practicing a medically proven form of contraception during the course of the study period. Written informed consent is obtained from each subject.

### Surgical Keloid Resection and Treatment Protocol

Ten to twelve patients will undergo bilateral keloid resection. Each patient will receive dermal administration of FLAP inhibitor compound formulated to an appropriate concentration of between 0.05 to 1.5% by weight in a clinically acceptable and safe topical formulation (solution, cream, ointment or gel) to each linear centimeter of one ear lobe wound margin immediately after wound closure and then repeatedly every 24 hours for 4 weeks. The other ear lobe will be treated topically with placebo (a clinically acceptable and safe topical formulation identical to that used in the treatment group, but lacking the active pharmaceutical ingredient) administered to each linear centimeter of ear lobe wound margin immediately after wound closure and then repeatedly every 24 hours for 4 weeks. The primary assessment is based on a photographic evaluation by a lay panel over a time period from week 4 to month 6 post surgery using a visual analog scale.

The primary outcome measure is to gain preliminary safety experience with the test compound in the keloid indication during the 52 week time frame. Secondary outcome measures are (i) reduction of keloid recurrence (Time frame 52 weeks) and (ii) physician global assessment and subject assessment (Time frame 52 weeks).

### Example 7

### Clinical Trial Evaluating Effect of a FLAP Inhibitor Compound on the Treatment and Prevention of Recurrence of Excised Keloids

### Treatment commencing 7 Days Post-Surgery

A double blind, randomized, placebo controlled within trial study to evaluate the safety and efficacy of a compound administered topically following the excision of a keloid scar on the ear lobe. Each patient undergoes bilateral keloid scar excision and one ear lobe is treated with compound while the other ear lobe is treated with placebo such that each patient will act as their own control.

Ten to twelve subjects aged 18-65 years are to participate in the study. All subjects should have bilateral keloid scars suitable for surgical excision such that, following excision, the result will be a single wound on each ear lobe no greater than 2 cm long. The wound will be restricted to the skin, fat and fibrous tissue of the ear lobe.

No subject should have experienced keloid treatment with irradiation, cryosurgery, corticosteroids or other pharmacological agents within 12 weeks prior to the study. Subjects should not have a history of a bleeding disorder and should not have experienced or have on-going psoriasis or eczema or malignant skin tumors. Female subjects of child bearing potential must have a documented negative urine pregnancy test and must be practicing a medically proven form of contraception during the course of the study period. Written informed consent is obtained from each subject.

### Surgical Keloid Resection and Treatment Protocol

Ten to twelve patients will undergo bilateral keloid resection. Each patient will receive dermal administration of a FLAP inhibitor compound formulated to an appropriate concentration of between 0.05 to 1.5% in a clinically acceptable and safe topical formulation (solution, cream, ointment or gel) to each linear centimeter of one ear lobe wound margin 7 days after wound closure and then repeatedly every 24 hours for 4 weeks. The other ear lobe will be treated topically with placebo (a clinically acceptable and safe topical formulation identical to that used in the treatment group, but lacking the active pharmaceutical ingredient) administered to each linear centimeter of ear lobe wound margin immediately after wound closure and then repeatedly every 24 hours for 4 weeks. The primary assessment is based on a photographic evaluation by a lay panel over a time period from week 4 to month 6 post surgery using a visual analog scale.

The primary outcome measure is to gain preliminary safety experience with the test compound in the keloid indication during the 52 week time frame. Secondary outcome measures are (i) reduction of keloid recurrence (Time frame 52 weeks) and (ii) physician global assessment and subject assessment (Time frame 52 weeks).

### Example 8

### Rabbit Wound Healing and Hypertrophic Scar Model

Following anesthesia, ear wounds are created in 10 young adult female New Zealand rabbits, 4 wounds per ear on each ear for a total of 8 wounds per animal. Wounds are created using a 7-mm biopsy punch with the wound created to go to bare cartilage. A dissecting microscope is used to ensure complete removal of the epidermis, dermis and perichondrium in each wound. For the hypertrophic scar model, it is the removal of the perichondrial layer and subsequent delay in reepithelialization of the defect that results in the elevated scar. Each wound heals independently and is considered a separate sample.

Two treatment groups are examined to study the early phase and a later phase of wound healing. The early treatment group (n=15 rabbits, 120 wounds) are treated with either the test compound formulated as a 0.05-1.5% by weight topical formulation (solution, cream, ointment or gel) or placebo using the topical vehicle formulation post-wounding on days 0, 1, 2, 3, 4, 5, 6 and 7 and harvested on day 28 after wounding. The later treatment group (n=15 rabbits, 120 wounds) are treated with either the test compound formulated as a 0.05-1.5% topical formulation (solution, cream, ointment or gel) or placebo using the topical vehicle formulation post-wounding on days 7, 8, 9, 10, 11, 12, 13 and 14 and harvested on day 28 after wounding. Half of the wounds in each group are treated with active compound and half are treated with placebo. Each wound is covered with a sterile dressing (Tegaderm; 3M) and dressings are changed daily following each treatment and as needed until the wound appears reepithelialized on gross examination. Wounds are excluded from analysis if there is evidence of infection, desiccation or necrosis.

At the end of each study wounds are harvested with a 5-mm margin of surrounding unwounded tissue. The scars are bisected and half of each wound is fixed in 4% neutral-buffered formaldehyde, dehydrated, embedded in paraffin, cut in 4-μm sections, and stained with Masson's trichrome or sirrus red. The other half of each wound is flash frozen in liquid nitrogen and stored for RNA extraction

### Histologic Analysis

Light microscopy is used to examine each tissue section and the degree of wound healing and scar hypertrophy are measured with a calibrated lens reticle in a blinded fashion. Wound healing parameters: Relevant measurements are granulation tissue ingrowth volume and height, wound epithelialization, and wound closure. Each parameter is assessed twice and the results are averaged.

Scar hypertrophy parameters: The scar elevation index is determined as described by Lu et al, J. Am. Coll. Surg., 2005, 201, p391-397. The values are determined twice by in a blinded fashion and the results averaged.

### Example 9

### Rat Abraded Skin Model to Test for Acute Irritancy and Skin Penetration Following Dermal Administration

Male Sprague Dawley Rats (n=3 per group) were shaved on the back such that approximately 20% of the body area surface was exposed. The shaved skin was mildly abraded then 2 mL of a 10 mg/mL solution of the test compound in a formulation containing 75% propylene glycol, 15% transcutol, 10% water was applied to the abraded skin. The site of administration was occluded for 24 hours after which time the skin was cleansed of excess material (if any). Plasma samples were drawn from each rat at 2, 4, 24 and 72 hours.

Visual analysis of erythema, edema or other dermal findings were recorded prior to dosing then at 24, 48 and 72 hours. Scoring followed a Draize protocol.

Results: Compound A (Na salt), Compound B (Na salt), Compound C (acid and Na salt), Compound F (Na salt), Compound G (Na salt), Compound J (Na salt), Compound I (acid), were all tested in the rat abraded skin model following dermal administration. All compounds were found to be negative in this study; a Draize score of 0 was recorded for each animal. PK analysis showed that all compounds had measurable plasma levels and that at the 24 hour time point the plasma levels were below 110 ng/mL.

### Example 10

### Mouse Arachidonic Acid-Induced Ear Inflammation Model

### Procedures

A mouse arachidonic acid (AA)—induced ear inflammation model was utilized to determine the extent of inhibition of ear swelling and leukotriene (LT) production by oral and topical application of a 5-lipoxygenase-activating protein (FLAP) inhibitor. Methods were adapted from Byrum et al., J Exp Med. 1997 Mar. 17; 185(6):1065-75).

Female CD-1 mice (weighing 18-20 grams) were administered compound orally (30 mg/kg in 10 ml/kg of 0.5% methylcellulose vehicle) or topically at concentrations of 0.01-10% by weight to the experimental ear and vehicle was applied to the opposite control ear. Four hours following compound administration a 40 μA aliquot of a 4 mg/ml solution of AA in ethanol was applied to the experimental (right) ear and vehicle (ethanol) was applied to the control (left) ear. Thirty minutes later mice were placed into an enclosed Plexiglas chamber and exposed to CO₂ for a period of 1-2 minutes or until breathing ceased. Ear biopsies were then taken for assessment of inflammation and leukotriene levels.

### Ear Inflammation

An eight mm ear punch was taken from a similar location of each experimental ear and each control ear and weighed. The extent of inflammation was determined by the difference in weight between the experimental ear and control ear [experimental ear (mg)-control ear (mg)]. A typical response for this assay is in the range of 4 and 12 mg increase in weight. Ear punches were then frozen for later assessment of leukotrienes.

### Leukotriene Analysis

The ear punch was placed into a tris buffer containing 0.01% triton x and homogenized using a polytron probe homogenizer. The sample was then centrifuged at 10,000×g for 10 minutes at 4° C. Leukotriene B₄ (LTB₄) and cysteinyl leukotriene (CysLT) concentrations in the supernatant were determined by enzyme-immunoassay (EIA).

### Results

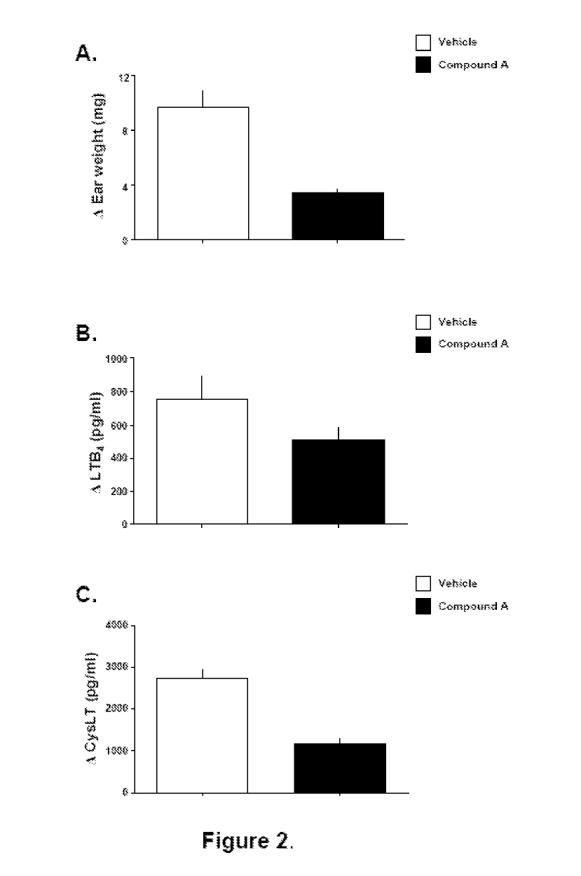

Oral dose study: AA caused pronounced ear inflammation with an increase in ear weight from 12 mg (control ear) to 21.7 mg (experimental ear). LT biosynthesis also increased with concentrations of CysLT in the supernatant increasing from 311 pg/ml to 3,051 pg/ml and LTB₄ showing a more modest increase of 245 pg/ml to 995 pg/ml. FIG. 2 illustrates the effect of the orally administered FLAP inhibitor Compound A on ear inflammation, LTB₄ and CysLT levels resulting from arachidonic acid-induced ear inflammation. Compound A given orally 4 hours prior to AA treatment, resulted in about 65% reduction of inflammation by measure of change in ear weight (FIG. 2A); about 33% reduction of LTB₄ levels (FIG. 2B); and about 57% reduction of CysLT levels (FIG. 2C).

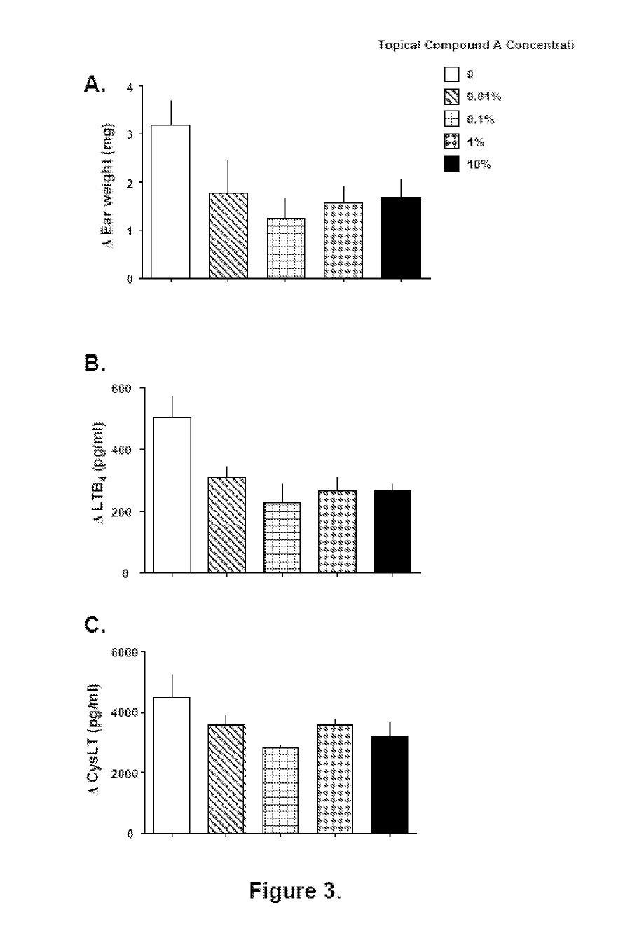

Dermal administration study: Similar to oral dose study, AA caused ear inflammation with an increase in ear weight from 12 mg (control ear) to 15.8 mg (experimental ear). Concentrations of CysLT in the supernatant increased from 399 pg/ml to 4,904 pg/ml and LTB₄ showed an increase of 236 pg/ml to 729 pg/ml. Compound A given topically at concentrations of 0.01-10% reduced all three endpoints with a maximal activity at 0.1%. FIG. 3 illustrates the effect of the dermally administered FLAP inhibitor Compound A on ear inflammation, LTB₄ and CysLT levels resulting from arachidonic acid-induced ear inflammation. COMPOUND A applied dermally 4 hours prior to AA treatment, resulted in about 60% reduction of inflammation by measure of change in ear weight (FIG. 3A); about 55% reduction of LTB₄ levels (FIG. 3B); and about 37% reduction of CysLT levels (FIG. 3C). The magnitude of inhibition following topical application was similar to that observed after oral dosing.

### Example 11

### Effects of DP2 Antagonist on Mucin Levels

BALB/c mice were divided into groups and acclimatized in cages for 24 hours (day 0). The control group was exposed to air and the test group was exposed to smoke from seven unfiltered cigarettes per day for 8 days (day 1 to day 8). FLAP inhibitor compound (3-(3-(tert-butylthio)-1-(4-(6-methoxypyridin-3-yl)benzyl)-5-((5-methylpyridin-2-yl)methoxy)-1H-indol-2-yl)-2,2-dimethylpropanoic acid) (30 mg/kg, b.i.d.), DP₂ receptor antagonist 5-{2-[(N-benzyloxycarbonyl-N-ethyl-amino)-methyl]-4-trifluoromethyl-phenyl}-pyridin-3-yl)-acetic acid (10 mg/kg qd), or a combination of FLAP inhibitor compound (3-(3-(tert-butylthio)-1-(4-(6-methoxypyridin-3-yl)benzyl)-5-((5-methylpyridin-2-yl)methoxy)-1H-indol-2-yl)-2,2-dimethylpropanoic acid) (30 mg/kg, b.i.d.) and DP₂ receptor antagonist 5-{2-[(N-benzyloxycarbonyl-N-ethyl-amino)-methyl]-4-trifluoromethyl-phenyl}-pyridin-3-yl)-acetic acid (10 mg/kg qd) are administered starting at day 1 and up to day 13. On day 14, bronchioalveolar lavage fluid (BALF) is tested for influx of cells, cytokines, chemokines (e.g., KC, IL-17, MIP-2, IL-6), mucin, and/or proteins. Lung histology is also examined. Trough plasma concentration is shown in the following table:

| | Plasma concentration |
|---|---|
| | (nM) @ trough |
| DP₂ receptor antagonist | <10 |
| 10 mg/kg, qd | |
| FLAP inhibitor | 168 |
| 30 g/kg, b.i.d. | |
| DP₂ receptor antagonist + | <10 (DP₂ receptor antagonist)/ |
| FLAP inhibitor | 136 (FLAP inhibitor) |

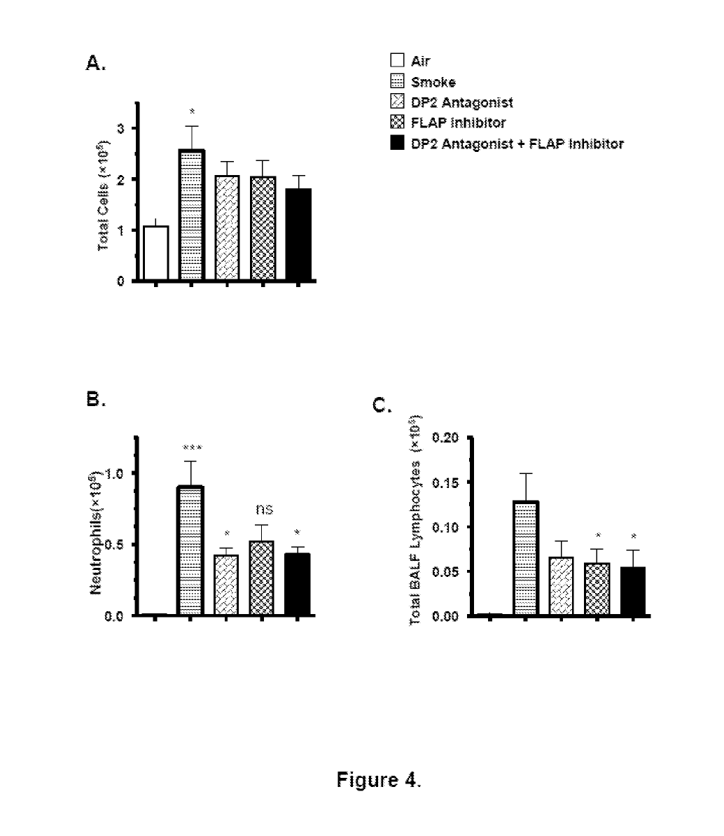

FIG. 4 illustrates the effect of FLAP inhibition, DP₂ receptor antagonism and combination of FLAP inhibition and DP₂ receptor antagonism on the number of total cells (FIG. 4A), neutrophils (FIG. 4B) and lymphocytes (FIG. 4C) present in BALF. In FIG. 4A, * represents P<0.05 vs. air, one-way ANOVA; in FIG. 4B, * is P<0.05 vs. smoke, one-way ANOVA with Tukey's, *** is P<0.001 vs. air, one-way ANOVA with Tukey's, “ns” is P=0.055, one-tailed t-test; and in FIG. 4C, * is P<0.05 vs. smoke, one-tailed t-test.

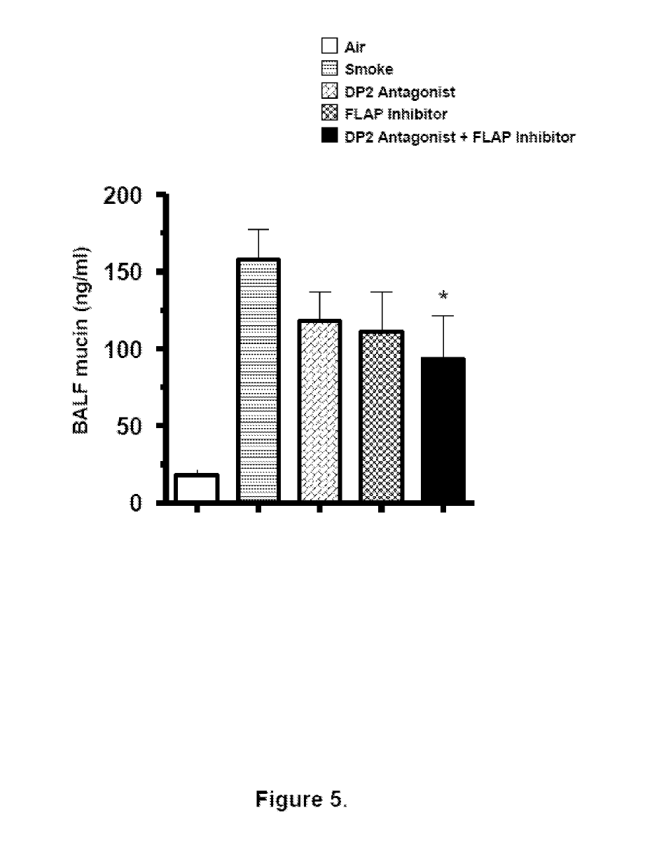

FIG. 5 illustrates the effect of a FLAP inhibitor, a DP₂ receptor antagonist and a combination of a FLAP inhibitor and a DP₂ receptor antagonist on the presence of mucin in BALF. In FIG. 5, * represents P<0.05 vs. smoke, one-tailed t-test. In the subchronic smoking mouse model, the effects of a combination of a FLAP inhibitor compound and a DP₂ receptor antagonist compound on mucin secretion in BALF were additive, i.e., a combination of a FLAP inhibitor compound and DP₂ receptor antagonist reduced the amount of mucin in BALF more than each compound alone. In one aspect, the effects of topical administration of a FLAP inhibitor compound, either alone or in combination with a DP₂ receptor antagonist, to the skin has the same effects (e.g., mechanistically expected) as observed in the BALF.

The examples and embodiments described herein are for illustrative purposes and various modifications or changes suggested to persons skilled in the art are to be included within the spirit and purview of this application and scope of the appended claims. The section headings used herein are for organizational purposes only and are not to be construed as limiting the subject matter described.

1. A topical formulation comprising a FLAP inhibitor compound in an amount effective for the treatment of a dermatological disease, disorder, or condition in a mammal, and at least one suitable pharmaceutically excipient to provide an ointment, cream, lotion, paste, gel, stick, a liposome, a nanoparticle, a patch or wound dressing.

2. The topical formulation of claim 1, wherein the FLAP inhibitor compound inhibits leukotriene synthesis, antagonizes a leukotriene receptor, inhibits Interleukin-4 (IL-4) synthesis, or inhibits mucin synthesis.

3. The topical formulation of claim 1, wherein the dermatological disease, disorder, or condition is an immune disorder; a proliferative disorder; contact with an allergen and/or an irritant, itch, atopic dermatitis, allergic dermatitis, bullous disorders, collagenoses, psoriasis, psoriatic lesions, contact dermatitis, eczema, urticaria, rosacea, hypertrophic scarring, keloid scar formation, scleroderma, Folliculitis keloidalis nuchae, Kawasaki Disease, Sjogren-Larsso Syndrome, Grover's disease, an overproduction of sebum lipids, acne, a first degree burn, a second degree burn, a third degree burn, a fourth degree burn, solar keratosis, squamous cell carcinoma or melanoma or combinations thereof.

4. The topical formulation of claim 1, wherein the FLAP inhibitor compound is a compound of Formula (I), or a pharmaceutically acceptable salt, or N-oxide thereof: 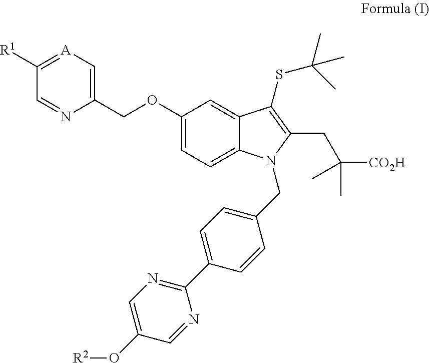
wherein,
A is CH or N;
R¹ is —F, —Cl, —Br, —CN, C₁-C₄alkyl, C₁-C₄fluoroalkyl, —O—C₁-C₄alkyl, or —O—C₁-C₄fluoroalkyl;
R² is C₁-C₄alkyl or C₁-C₄fluoroalkyl.

5. The topical formulation of claim 4, wherein the FLAP inhibitor compound is 3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid (Compound A); or 3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid (Compound B), or a pharmaceutically acceptable salt, or N-oxide thereof.

6. The topical formulation of claim 1, wherein the FLAP inhibitor compound is a compound of Formula (II), or a pharmaceutically acceptable salt, or N-oxide thereof: 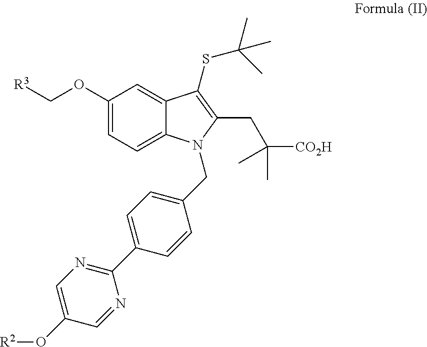
wherein,
R² is C₁-C₄alkyl or C₁-C₄fluoroalkyl;
R³ is a substituted or unsubstituted monocyclic or bicyclic heterocycloalkyl.

7. The topical formulation of claim 6, wherein R³ is selected from the group consisting of: 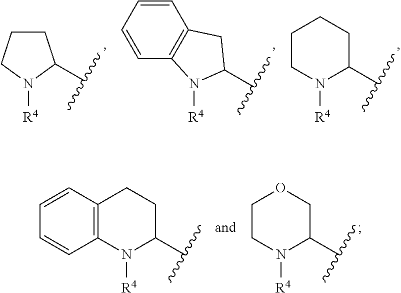
R⁴ is H, —C(═O)R⁵ or —SO₂—C₁-C₄alkyl; R⁵ is C₁-C₄alkyl, C₁-C₄fluoroalkyl, substituted or unsubstituted phenyl, substituted or unsubstituted heteroaryl, or —O—C₁-C₄alkyl.

8. The topical formulation of claim 7, wherein the compound of Formula (II) has the following structure:

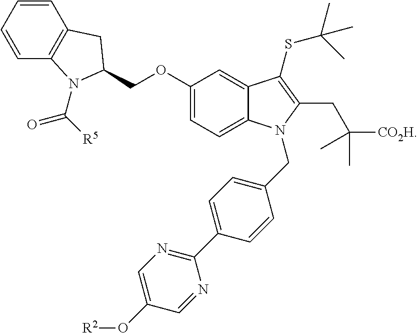

9. The topical formulation of claim 8, wherein the FLAP inhibitor compound is 3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid (Compound C), 3-{5-((S)-1-Acetyl-2,3-dihydro-1H-indol-2-ylmethoxy)-3-tert-butyl sulfanyl-1-[4-(5-ethoxy-pyrimidin-2-yl)-benzyl]-1H-indol-2-yl}-2,2-dimethyl-propionic acid (Compound K), or a pharmaceutically acceptable salt, or N-oxide thereof.

10. The topical formulation of claim 1, wherein the FLAP inhibitor compound is selected from: 3-[3-tert-butylsulfanyl-1-(4-chloro-benzyl)-5-isopropyl-1H-indol-2-yl]-2,2-dimethyl-propionic acid (MK886); 3-[3-tert-butylsulfanyl-1-(4-chloro-benzyl)-5-(quinolin-2-ylmethoxy)-1H-indol-2-yl]-2,2-dimethyl-propionic acid (MK591); cyclopentyl-[4-(quinolin-2-ylmethoxy)-phenyl]-acetic acid (DG031; BAY X1005); 3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid (Compound A); 3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid (Compound B); 3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid (Compound C); 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid (Compound D); 3-[3-tert-Butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid (Compound E); 3-[3-tert-Butylsulfanyl-1-[4-(5-fluoro-pyridin-2-yl)-benzyl]-5-(quinolin-2-ylmethoxy)-1H-indol-2-yl]-2,2-dimethyl-propionic acid (Compound F); 2-[3-tert-Butylsulfanyl-1-[4-(5-methoxy-pyrimidin-2-yl)-benzyl]-5-(5-methyl-pyridin-2-ylmethoxy)-1H-indol-2-ylmethyl]-2-ethyl-butyric acid (Compound G); 3-[3-tert-Butylsulfanyl-1-[4-(6-methoxy-pyridin-3-yl)-benzyl]-5-(5-methyl-pyridin-2-ylmethoxy)-1H-indol-2-yl]-2,2-dimethyl-propionic acid (Compound H); 3-[5-((S)-1-Acetyl-pyrrolidin-2-ylmethoxy)-3-tert-butylsulfanyl-1-(4-chloro-benzyl)-1H-indol-2-yl]-2,2-dimethyl-propionic acid (Compound I); 3-[3-tert-butylsulfanyl-1-[4-(5-fluoro-pyridin-2-yl)-benzyl]-5-(pyridin-2-ylmethoxy)-1H-indol-2-yl]-2,2-dimethyl-propionic acid (Compound J); 3-{5-((S)-1-Acetyl-2,3-dihydro-1H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid (Compound K); or a pharmaceutically acceptable salt, or N-oxide thereof.

11. The topical formulation of claim 1, further comprising a therapeutically-effective amount of an second compound, wherein the second compound is an antibiotic; anti-fungal agent; steroid anti-inflammatory agent; non-steroidal anti-inflammatory agent; antihistamine; antiviral; alpha agonist; beta blocker; carbonic anhydrase inhibitor; miotic; prostaglandin; anti-angiogenesis agent; loteprednol etabonate, mast cell stabilizer, cyclosporine, or DP₂ antagonist.

12. A method of treating of a dermatological disease, disorder, or condition, comprising administering to an individual in need thereof a therapeutically-effective amount of a topical formulation comprising a FLAP inhibitor compound.

13. The method of claim 12, wherein the FLAP inhibitor compound inhibits leukotriene synthesis, antagonizes a leukotriene receptor, inhibits Interleukin-4 (IL-4) synthesis, or inhibits mucin synthesis.

14. The method of claim 12, wherein the topical formulation is in the form of an ointment, cream, lotion, paste, gel, stick, a liposome, a nanoparticle, a patch or wound dressing.

15. The method of claim 12, wherein the FLAP inhibitor compound is a compound of Formula (I), or a pharmaceutically acceptable salt, or N-oxide thereof: 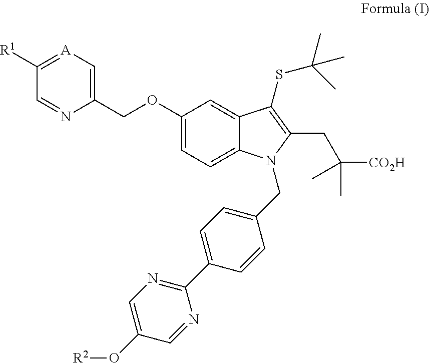
wherein,
A is CH or N;
R¹ is —F, —Cl, —Br, —CN, C₁-C₄alkyl, C₁-C₄fluoroalkyl, —O—C₁-C₄alkyl, or —O—C₁-C₄fluoroalkyl;
R² is C₁-C₄alkyl or C₁-C₄fluoroalkyl.

16. The method of claim 15, wherein the FLAP inhibitor compound is 3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid (Compound A); or 3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid (Compound B), or a pharmaceutically acceptable salt, or N-oxide thereof.

17. The method of claim 12, wherein the FLAP inhibitor compound is a compound of Formula (II), or a pharmaceutically acceptable salt, or N-oxide thereof: 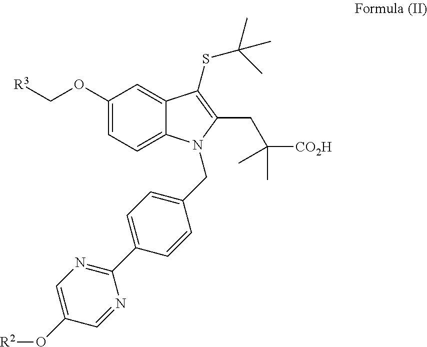
wherein,
R² is C₁-C₄alkyl or C₁-C₄fluoroalkyl;
R³ is a substituted or unsubstituted monocyclic or bicyclic heterocycloalkyl.

18. The method of claim 17, wherein R³ is selected from the group consisting of: 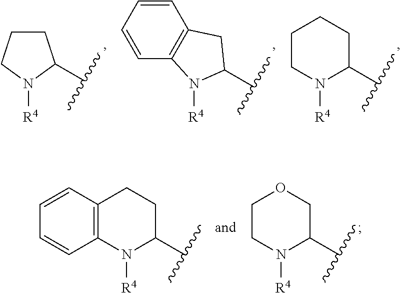
R⁴ is H, —C(═O)R⁵ or —SO₂—C₁-C₄alkyl; R⁵ is C₁-C₄alkyl, C₁-C₄fluoroalkyl, substituted or unsubstituted phenyl, substituted or unsubstituted heteroaryl, or —O—C₁-C₄alkyl.

19. The method of claim 18, wherein the compound of Formula (II) has the following structure:

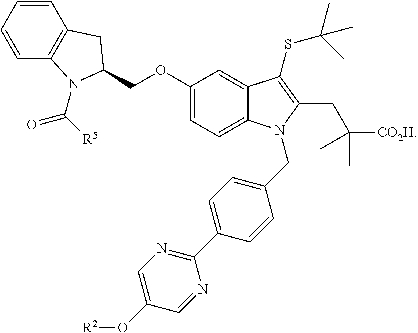

20. The method of claim 19, wherein the FLAP inhibitor compound is 3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid (Compound C), 3-{5-((S)-1-Acetyl-2,3-dihydro-1H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid (Compound K), or a pharmaceutically acceptable salt, or N-oxide thereof.

21. The method of claim 12, wherein the FLAP inhibitor compound is selected from: 3-[3-tert-butylsulfanyl-1-(4-chloro-benzyl)-5-isopropyl-1H-indol-2-yl]-2,2-dimethyl-propionic acid (MK886); 3-[3-tert-butylsulfanyl-1-(4-chloro-benzyl)-5-(quinolin-2-ylmethoxy)-1H-indol-2-yl]-2,2-dimethyl-propionic acid (MK591); cyclopentyl-[4-(quinolin-2-ylmethoxy)-phenyl]-acetic acid (DG031; BAY X1005); 3-[3-tert-Butylsulfanyl-1-[4-(5-methoxy-pyrimidin-2-yl)-benzyl]-5-(5-methyl-pyridin-2-ylmethoxy)-1H-indol-2-yl]-2,2-dimethyl-propionic acid (Compound A); 3-[3-tert-Butylsulfanyl-1-[4-(5-methoxy-pyrimidin-2-yl)-benzyl]-5-(5-methyl-pyrazin-2-ylmethoxy)-1H-indol-2-yl]-2,2-dimethyl-propionic acid (Compound B); 3-{5-((S)-1-Acetyl-2,3-dihydro-1H-indol-2-ylmethoxy)-3-tert-butylsulfanyl-1-[4-(5-methoxy-pyrimidin-2-yl)-benzyl]-1H-indol-2-yl}-2,2-dimethyl-propionic acid (Compound C); 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid (Compound D); 3-[3-tert-Butylsulfanyl-1-[4-(6-ethoxy-pyridin-3-yl)-benzyl]-5-(5-methyl-pyridin-2-ylmethoxy)-1H-indol-2-yl]-2,2-dimethyl-propionic acid (Compound E); 3-[3-tert-Butylsulfanyl-1-[4-(5-fluoro-pyridin-2-yl)-benzyl]-5-(quinolin-2-ylmethoxy)-1H-indol-2-yl]-2,2-dimethyl-propionic acid (Compound F); 2-[3-tert-Butylsulfanyl-1-[4-(5-methoxy-pyrimidin-2-yl)-benzyl]-5-(5-methyl-pyridin-2-ylmethoxy)-1H-indol-2-ylmethyl]-2-ethyl-butyric acid (Compound G); 3-[3-tert-Butylsulfanyl-1-[4-(6-methoxy-pyridin-3-yl)-benzyl]-5-(5-methyl-pyridin-2-ylmethoxy)-1H-indol-2-yl]-2,2-dimethyl-propionic acid (Compound H); 3-[5-((S)-1-Acetyl-pyrrolidin-2-ylmethoxy)-3-tert-butylsulfanyl-1-(4-chloro-benzyl)-1H-indol-2-yl]-2,2-dimethyl-propionic acid (Compound I); 3-[3-tert-butylsulfanyl-1-[4-(5-fluoro-pyridin-2-yl)-benzyl]-5-(pyridin-2-ylmethoxy)-1H-indol-2-yl]-2,2-dimethyl-propionic acid (Compound J); 3-{5-((S)-1-Acetyl-2,3-dihydro-1H-indol-2-ylmethoxy)-3-tert-butylsulfanyl-1-[4-(5-ethoxy-pyrimidin-2-yl)-benzyl]-1H-indol-2-yl}-2,2-dimethyl-propionic acid (Compound K); or a pharmaceutically acceptable salt, or N-oxide thereof.

22. The method of claim 12, wherein the dermatological disorder is an immune disorder; a proliferative disorder; contact with an allergen and/or an irritant, itch, atopic dermatitis, allergic dermatitis, bullous disorders, collagenoses, psoriasis, psoriatic lesions, contact dermatitis, eczema, urticaria, rosacea, hypertrophic scarring, keloid scar formation, scleroderma, Folliculitis keloidalis nuchae, Kawasaki Disease, Sjogren-Larsso Syndrome, Grover's disease, an overproduction of sebum lipids, acne, a first degree burn, a second degree burn, a third degree burn, a fourth degree burn, solar keratosis, squamous cell carcinoma or melanoma.

23. The method of claim 12, wherein the dermatological disease, disorder, or condition is scarring.

24. The method of claim 23, wherein the scarring results in the formation of a keloid scar.

25. The method of claim 12, wherein the dermatological disease, disorder, or condition results from surgery and the topical formulation is administered before or after surgery.

26. The method of claim 12, wherein the topical formulation is administered before or after contact with an irritant and/or allergen.

27. The method of claim 12, further comprising administering to the individual in need thereof a therapeutically-effective amount of a second compound, wherein the second compound is an antibiotic; anti-fungal agent; steroid anti-inflammatory agent; non-steroidal anti-inflammatory agent; antihistamine; antiviral; alpha agonist; beta blocker; carbonic anhydrase inhibitor; miotic; prostaglandin; anti-angiogenesis agent; loteprednol etabonate, mast cell stabilizer, cyclosporine, or DP₂ antagonist.
